(19) United States
(12) Patent Application Publication
(10) Pub. No.: US 2025/0221674 A1
(43) Pub. Date: Jul. 10, 2025

(54) SYSTEMS, DEVICES, AND METHODS FOR WELLNESS MONITORING WITH PHYSIOLOGICAL SENSORS

(71) Applicant: LINGO SENSING TECHNOLOGY UNLIMITED COMPANY, Dublin 2 (IE)

(72) Inventors: Justin N. Williams, Oakland, CA (US); Jessica Inchauspe, New York, NY (US); James P. McCarter, St. Louis, MO (US); Junli Ou, Pleasanton, CA (US)

(21) Appl. No.: 18/724,471

(22) PCT Filed: Dec. 27, 2022
(86) PCT No.: PCT/US2022/054054

§ 371 Date: Jun. 26, 2024

(51) Int. Cl. A61B 5/00; A61B 5/145; G16H 40/67

(52) U.S. Cl. CPC A61B 5/743; A61B 5/0004; A61B 5/14532; A61B 5/7425; A61B 5/7435; G16H 40/67

(57) ABSTRACT

Systems and methods for monitoring glucose variability in a subject are described. Data indicative of glucose levels of the subject is received from a sensor control device. A first glucose variability metric of the subject in a first time period is determined. The first glucose variability metric may be compared to a threshold. A first indicator is displayed if the first glucose variability metric does not exceed the threshold and a second indicator is displayed if the first glucose variability metric exceeds the threshold. Additional glucose variability metrics may be determined for subsequent time periods according to a rolling window, and the indicators may be displayed real time or in a report. The glucose variability metric may be a measure of variability compared to a baseline, a difference between a maximum and minimum glucose level, or time in or out of a target range.

### CROSS-REFERENCE TO RELATED APPLICATIONS

This application is a national stage patent application under 35 U.S.C. § 371 of International Application No. PCT/US2022/054054, filed Dec. 27, 2022, which claims priority to U.S. Provisional Application No. 63/337,442, filed May 2, 2022, and U.S. Provisional Application No. 63/295,284, filed Dec. 30, 2021, all of which are herein expressly incorporated by reference in their entireties for all purposes.

### FIELD

The subject matter described herein relates generally to digital interfaces and user interfaces for analyte monitoring systems, as well as systems, methods, and devices relating thereto.

### BACKGROUND

The detection and/or monitoring of analyte levels, such as glucose, ketones, ketone bodies (e.g., β-hydroxybutyrate), lactate, oxygen, hemoglobin A1C, or the like, can be vitally important to the overall health of a person, particularly for an individual having diabetes. Patients suffering from diabetes mellitus can experience complications including loss of consciousness, cardiovascular disease, retinopathy, neuropathy, and nephropathy. Persons with diabetes are generally required to monitor their glucose levels to ensure that they are being maintained within a clinically safe range, and may also use this information to determine if and/or when insulin is needed to reduce glucose levels in their bodies, or when additional glucose is needed to raise the level of glucose in their bodies.

Growing clinical data demonstrates a strong correlation between the frequency of glucose monitoring and glycemic control. Despite such correlation, however, many individuals diagnosed with a diabetic condition do not monitor their glucose levels as frequently as they should due to a combination of factors including convenience, testing discretion, pain associated with glucose testing, and cost.

To increase patient adherence to a plan of frequent glucose monitoring, in vivo analyte monitoring systems can be utilized, in which a sensor control device may be worn on the body of an individual who requires analyte monitoring. To increase comfort and convenience for the individual, the sensor control device may have a small form-factor and can be applied by the individual with a sensor applicator. The application process includes inserting at least a portion of a sensor that senses a user's analyte level in a bodily fluid located in a layer of the human body, using an applicator or insertion mechanism, such that the sensor comes into contact with the bodily fluid. The analyte monitoring system may also be configured to transmit analyte data and/or alarms to another device, from which a caregiver such as, for example, a parent, a spouse, or a health care provider (“HCP”), can review the data and make therapy decisions. Furthermore, the benefits of analyte monitoring systems are not limited to persons with diabetes. For instance, analyte monitoring systems can provide useful information and insights to individuals interested in improving their health and wellness. As one example, to improve their sports performance, athletes can utilize a sensor control device worn on the body to collect data relating to one or more analytes such as, for example, glucose and/or lactate. Other non-medical applications for analyte monitoring systems are possible and described in further detail below.

Despite their advantages, however, some people are reluctant to use analyte monitoring systems for various reasons, including the complexity and volume of data presented, a learning curve associated with the software and user interfaces for analyte monitoring systems, and an overall paucity of actionable information presented.

Furthermore, as sensor control devices have become more convenient, comfortable, and affordable for users, applications outside of medicine have become feasible. For example, high-performance athletes are interested in optimizing levels of performance-affecting analytes, for example blood glucose, before or during training and competition. However, some existing user interfaces for sensor control devices are designed for medical use by patients under care of a physician, and not for non-medical applications such as, for example, athletic training and competition. As such, the data collected by the sensor control device, and methods for presenting the data to the user, may be unsuitable for non-medical applications. In addition, sensor control devices for non-medical (e.g., wellness and fitness) use may be confused with similar devices made for medical use, leading to problems in interpreting or using data.

Various applications make use of the sensor data to perform various functions, including wellness functions. However, each software that desires to use the sensor data may become subject to regulatory standards or require regulatory clearance and be viewed as software as a medical device. Any new application desiring to make a use-case for the physiological data that can be obtained from sensors may face regulatory hurdles under the Food and Drug Administration. Thus, there exists a need to provide a framework that can communicate with physiological sensors and receive analyte data for use by various applications, including third party applications, but avoids the need for regulatory approval for every use-case for the data. Moreover, a need exists for digital interfaces and graphical user interfaces for analyte monitoring systems for medical and/or non-medical use, as well as methods and devices relating thereto, that are robust, user-friendly, and provide for timely and actionable responses.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter is directed to a software library for use by applications to obtain sensor data. The software library can include a sensor control module, a remote management module, and include software logic for communication with a plurality of physiological sensors and applications. The sensor control module can authenticate the receiving device to allow the receiving device to receive sensor data, including by enabling communication with each of the plurality of physiological sensors to receive sensor data including data indicative of a different physiological signal. The sensor control module can further store the sensor data in a memory of the computing device. The sensor control module can obtain an output indicative of the different physiological signals from the sensor data of each of the plurality of physiological sensors. The sensor control module can provide the output of the different physiological signals from the physiological sensors to the authenticated third-party application running on the computing device.

In accordance with the disclosed subject matter, the physiological sensors can comprise an analyte sensor configured to detect an analyte level in a bodily fluid of a user. The output of the different physiological signals can also comprise an analyte value. The output can further comprise a notification of a physiological condition. The output can further indicate information about delivery of a medicament to a user.

In accordance with the disclosed subject matter, the communication session within the computing device and between the computing device and the physiological sensors can comprise a near-field communication (NFC), Bluetooth low energy (BLE), or any suitable wireless communication protocol known in the art.

The software library can further include a remote data management module including instructions to transmit sensor data to a remote server over a network. The remote management module can be configured to communicate with the remote server to authenticate the sensor control module, third-party application, or any other application. The authentication can use a uniform user interface irrespective of the application accessing the software library.

In accordance with the disclosed subject matter, the plurality of physiological sensors and the software library are subject to regulatory approval, including as software as a medical device. The output indicative of the physiological signal from the physiological sensors is also subject to regulatory approval. However, the third-party application running on the computing device is not subject to regulatory approval.

The software library can be configured to be implemented as a component of the authenticated third-party application. Because of the modular architecture and shared functionality, sensor data can be substantially simultaneously received, interpreted, and displayed from a plurality of physiological sensors.

In accordance with the disclosed subject matter, a method for monitoring glucose variability includes a system that receives data indicative of glucose levels of the subject from a sensor control device is described. A first glucose variability metric of the subject may be determined in a first time period. The first glucose variability metric may then be compared to a threshold. A first indicator may be displayed if the first glucose variability metric does not exceed the threshold and a second indicator may be displayed if the first glucose variability metric exceeds the threshold.

The glucose variability metric may be variability with respect to a running baseline, a difference between a maximum and minimum glucose level, time in or out of a target range during the relevant time period, or a combination thereof.

In accordance with the disclosed subject matter, a method for monitoring glucose variability includes a system that receives data indicative of glucose levels of the subject from a sensor control device is described. A maximum glucose level and a minimum glucose level in a time period may be identified. A difference of the maximum glucose level and the minimum glucose level in the time period may be calculated. The difference may be compared to a threshold. A first indicator may be displayed if the difference does not exceed the threshold and a second indicator may be displayed if the difference exceeds the threshold.

In accordance with the disclosed subject matter, a system for displaying metrics relating to a subject is described. The system includes one or more processors and a memory storing instructions that, when executed by the one or more processors, cause the system to: determine a glucose status of the subject based on glucose data received in a rolling window time period; display an indication of the glucose status of the subject in a graphic user interface (GUI), wherein the indication of the glucose status comprises a text description and a graphic having a first color; and display a graph in the GUI, wherein the graph comprises a glucose profile comprising a first portion and a second portion, wherein the first portion and second portion are different colors, and wherein the second portion is the first color.

In accordance with the disclosed subject matter, a system for displaying metrics relating to a subject is described. The system includes one or more processors and a memory storing instructions that, when executed by the one or more processors, cause the system to: determine a plurality of glucose statuses of the subject based on glucose data received in a plurality of rolling window time periods, wherein each of the rolling window time periods comprises a logged activity; display a first graph comprising a first glucose profile for a first rolling window time period and a description of a first logged activity, wherein the first glucose profile comprises first, second, and third portions, wherein the first portion and third portions are a first color, and wherein the second portion is a second color, and display a second graph comprising a second glucose profile for a second rolling window time period and a description of a second logged activity, wherein the second glucose profile comprises first, second, and third portions, wherein the first portion and third portions are the first color, and wherein the second portion is a third color.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a system overview of a system that includes a software library, receiving device, and sensor assembly.

FIG. 2 is a block diagram depicting an example embodiment of a receiving device.

FIG. 3 is a block diagram depicting an example embodiment of a sensor assembly.

FIG. 4 is a block diagram depicting an example software library, including a sensor control module and a remote management module, for communication with applications.

FIG. 5 is a block diagram depicting an example embodiment of the sensor control module.

FIG. 6 is a block diagram depicting an example embodiment of the remote management module.

FIG. 7A-7C are exemplary embodiments of user interfaces of applications using the inventive architecture.

FIGS. 8-9 are example methods for communicating sensor data from a sensor to an application or a third-party application using the disclosed subject matter.

FIGS. 10A-10E are example embodiments of GUIs related to a biosensor banner.

FIGS. 11A-11B are example embodiments of GUIs related to a biosensor module details.

FIGS. 12A-12B are example embodiments of GUIs related to system messages associated with a biosensor.

FIGS. 13A-13D are block diagrams depicting example embodiments of GUIs related to pairing a biosensor with a reader device.

FIG. 14 is a system overview of an analyte monitoring system comprising a sensor applicator, a sensor control device, a reader device, a network, a trusted computer system, and a local computer system.

FIG. 15A is a block diagram depicting an example embodiment of a reader device.

FIGS. 15B and 15C are block diagrams depicting example embodiments of sensor control devices.

FIGS. 16A-16B are block diagrams depicting example embodiments of GUIs related to live home screens.

FIGS. 17A-17B are block diagrams depicting example embodiments of GUIs related to insight screens with further details.

FIG. 18 is a block diagram depicting an example embodiment of a GUI related to sharing the user's progress.

FIG. 19 is a block diagram depicting an example embodiment of a GUI related to tracking different activities.

FIG. 20 is a block diagram depicting an example embodiment of a GUI related to learning and exploring. exposure.

FIGS. 21A-21C are exemplary methods of determining glucose variability.

### DETAILED DESCRIPTION

Reference will now be made in detail to the various exemplary embodiments of the disclosed subject matter, exemplary embodiments of which are illustrated in the accompanying drawings.

The system can include a device that receives analyte data measured by an analyte monitor and medication delivery data recorded by a delivery device, and processes and/or displays that data, in any number of forms, to the user. This device, and variations thereof, can be referred to as a “receiving device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. This device can be a smartphone, a smartwatch, or display device.

The system can also include an in vivo analyte monitor sensor assembly, which can comprise various types of monitors. For example, “Continuous Analyte Monitoring” systems (or “Continuous Glucose Monitoring” systems), can transmit data from a sensor device to a reader device continuously without prompting, e.g., automatically according to a schedule. “Flash Analyte Monitoring” systems (or “Flash Glucose Monitoring” systems or simply “Flash” systems), as another example, can transfer data from a sensor device in response to a scan or request for data by a reader device, such as with a Bluetooth Low-Energy (BLE), Near Field Communication (NFC) or Radio Frequency Identification (RFID) protocol. An in vivo analyte monitoring sensor assembly can also operate without the need for finger stick calibration.

In vivo monitoring sensor assemblies can include a sensor that, while positioned in vivo, contacts the bodily fluid of the user and generates analyte data indicative of the analyte levels contained therein. The sensor assembly can reside on the body of the user and contain the electronics and power supply that enable and control the analyte sensing. The sensor assembly, and variations thereof, can also be referred to as an “on-body electronics” device or unit, an “on-body” device or unit, or a “sensor data communication” device or unit, or analyte sensor, sensor device, in vivo analyte monitor sensor assembly, sensors, to name a few.

Further, the system can include an external device for use with the analyte sensor. For example and without limitation, external devices can include delivery devices that use information from the analyte sensor to determine or deliver amounts of a medication or other beneficial agents to a user. Additionally or alternatively, external devices can include other sensors, such as other analyte sensors, accelerometers, pressures sensors, or can include external computing devices, such as a medical server or a smartphone application configured to use analyte sensor information to provide additional insights to a user, including but not limited to insights related to medical conditions, well-being, fitness, appetite, or other medical or non-medical insights or analysis.

Generally, and as set forth in greater detail below, the disclosed subject matter provided herein includes a software library within a receiving device for communicating with analyte sensors and permitting third-party applications access to the sensor data for use in medically necessary applications or applications related to the well-being of the user. The system further includes a software library that can be implemented independently of the sensors and integrated within third-party applications to allow access to the sensor data. The sensor control module can further communicate with the sensor assemblies in such a manner to receive data simultaneously or substantially simultaneously from a plurality of such sensor assemblies. The system further enables the transfer of sensor information from the sensor control module to a remote management module.

The embodiments described herein can be used to monitor and/or process information regarding any number of one or more different analytes. Analytes that may be monitored include, but are not limited to, acetyl choline, amylase, bilirubin, cholesterol, chorionic gonadotropin, glycosylated hemoglobin (HbAlc), creatine kinase (e.g., CK-MB), creatine, creatinine, DNA, fructosamine, glucose, glucose derivatives, glutamine, growth hormones, hormones, ketones, ketone bodies (e.g., β-hydroxybutyrate), lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In embodiments that monitor more than one analyte, the analytes may be monitored at the same or different times.

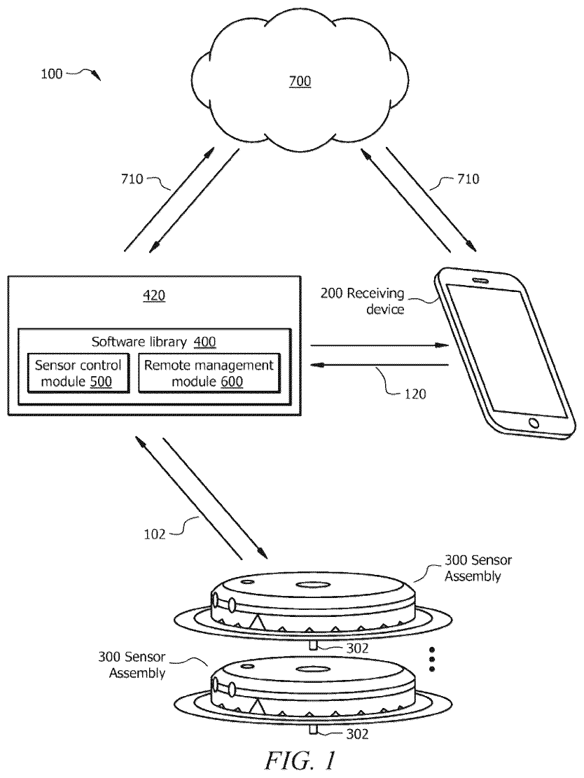

FIG. 1 is a schematic diagram depicting an example embodiment of a system 100 that includes a modular connectivity framework using a software library 400, various applications 420, a sensor assembly 300, and a receiving device 200.

In accordance with the disclosed subject matter, a non-transitory computer-readable storage medium includes a software library for use by applications 420 on a receiving device 200, or standalone devices such as a pump, insulin pen, etc., to obtain sensor data. The software library can include a sensor control module, a remote management module, and include software logic for communication with a plurality of sensors and applications. The sensor control module can authenticate the receiving device to allow the receiving device to receive sensor data, including by enabling communication with each of the plurality of sensors to receive sensor data including data indicative of a different signal. The sensor control module can further store the sensor data in a memory of the computing device. The sensor control module can obtain an output indicative of the different signals from the sensor data of each of the plurality of sensors. The sensor control module can provide the output of the different signals from the sensors to the authenticated third-party application running on the computing device.

The system 100 includes a software library 400 that functions using a modular architecture enabling a sensor control module 500 to communicate with and reside within various applications 420 on the receiving device 200. Applications 420 may further interface with sensor assembly 300 through the sensor control module 500, and in particular, by providing the request to the communication control module 540 (on FIG. 5) to interface directly with the sensor assembly 300. The sensor assembly 300 can also be one device with different sensors 302 or one sensor 302 configured to detect more than one analyte.

The receiving device 200 includes one or more applications 420, with each application instance embedding software library 400. The receiving device 200 uses a modular connectivity framework for the applications 420. In particular, the applications 420 each include a software library 400 including a remote management module 600 and sensor control module 500 for communicating with the one or more sensor assemblies 300. The software library 400 may also run as a service that executes simultaneously with the underlying application allowing the sensor control module 500 or remote management module 600 to execute as a service alongside one or more applications.

Sensor control module 500 may further interface with the sensor data. The various modules within the software library 400 implemented within the application 420 can send and receive communication with the sensor assembly 300 via communication link 102.

While the sensor control module 500 is within the application 420 in a receiving device 200, the sensor control module 500 could have base components in a second receiving device, such as a smartwatch, mobile device, or other wearable device. While such a device may not allow for a user interface experience that would be provided by a smartphone or tablet or computer, the smartwatch or wearable device can incorporate the sensor control module 500 to permit direct communication through the sensor control module 500 on the smartwatch or mobile wearable device with the sensor assembly 300. This would allow for applications specific to wearable devices to use sensor data. The wearable devices can synch separately with the receiving device 200, which can be used to perform the majority of the user login, initialization, authentication, and consent features to implement and initiate the receipt of sensor data.

Communication link 102 can be a wireless protocol including Bluetooth®, Bluetooth® Low Energy (BLE, BTLE, Bluetooth® SMART, etc.), Near-Field Communication (NFC) and others. The communication links 102 can each use the same or different wireless protocols. The system 100 may be configured to communicate over other wireless data communication links such as, but not limited to, RF communication link, infrared communication link, or any other type of suitable wireless communication connection between two or more electronic devices, which may further be uni-directional or bi-directional communication. Alternatively, the data communication link may include wired cable connection such as, for example, but not limited to, RS232 connection, USB connection, FireWire, Lightning, or serial cable connection.

For example, and as embodied herein, communication link 102 can be configured to use a Bluetooth protocol, such as BLE, or communication link 102 can be configured to use an NFC protocol. Additionally or alternatively, another communication link not shown may exist between a second sensor assembly and it can be configured to use BLE or both NFC and BLE. The communication links can be configured to perform different operations. For example, communication link 102 can be configured to perform only activation of the sensor assembly. Furthermore, communication links can have different configurations depending on the overall system architecture or the components that are activated or being used in the system at a given time. For example, and as embodied herein, communication link 102 can have a first communication configuration when the receiving device 200 is active in the system and a second communication configuration when the receiving device is not active or not included in the system.

In the first communication configuration, the communication link 102 can be configured only to perform activation of the sensor using an NFC wireless protocol. In another configuration, BLE capability (if provided) can remain inactive between the sensor assembly 300 and the applications 420. The application 420 can activate the sensor assembly 300 using NFC wireless protocol and obtain sensor context information. Sensor context information can include authentication information for authenticating a communication session with the sensor assembly 300, encryption information to enable encrypted data communication over the communication links, and a BLE communication address to initiate a BLE connection with the sensor assembly 300. The software library 400 may also obtain the sensor context information from the sensor assembly 300 over BLE. Using the sensor context information, the software library 400 includes capabilities to allow a session to switch from an application 420 on the receiving device 200 such as a smartphone to another application 420 on another receiving device 200 such as a smartwatch. The sensor context information can be transmitted within the applications 420.

In accordance with the disclosed subject matter, the sensor assembly 300 as shown may include sensing elements for detecting different analytes within the same sensor assembly. The system 100 may also include multiple sensor assemblies 300, as shown, connected via a communication link having similar capabilities of communication to the communication link 102 described herein. Two or more sensor assemblies 300 can also be used in conjunction by having multiple sensing elements that together produce the reading for an analyte, or separately produce readings for different analytes. Any number of sensor assemblies could be used together to measure any number of different analyte values, and two sensor assemblies are shown for illustration, not limitation, in this disclosure.

In some embodiments, the application 420 can be configured to access the software library 400 through a remote cloud 700 infrastructure via wireless communication links 710. In certain embodiments, the communication link 710 includes a wireless communication section configured for bi-directional radio frequency (RF) communication with other devices to transmit and/or receive data to and from the system 100. In addition, the communication link 710 may also be configured to include physical ports or interfaces such as one or more of a USB port, an RS-232 port, a serial port, a IEEE 1394 (Firewire) port, an Ethernet port or any other suitable electrical connection port to allow data communication between the system 100 and receiving device 200, such as a personal computer, a laptop computer, a notebook computer, an iPad, a tablet computing device, a cellular telephone, a smart phone, a personal data assistant, a workstation, a server, a mainframe computer, a cloud computing system, an external medical device, such as an infusion device, an analyte monitoring device, or including an insulin delivery device, or other devices that are configured for similar complementary data communication. In certain embodiments, communication link 710 may include a cellular communication protocol, a Wi-Fi (IEEE 802.1x) communication protocol, or an equivalent wireless communication protocol which would allow secure, wireless communication of several units (for example, per HIPPA requirements) while avoiding potential data collision and interference.

In other embodiments, the wireless communication section 710 may be configured for infrared communication, Bluetooth communication, wireless USB communication, ZigBee communication, cellular communication, Wi-Fi (IEEE 802.11x) communication, RFID (passive or active) communication, or any other suitable wireless communication mechanism to enable the receiving device 200 to communicate with other devices such as infusion devices, analyte monitoring devices, computer terminals, servers, personal computers, laptop computers, notebook computers, iPads, tablet computers, cell phones, smart phones, workstations, mainframe computers, cloud computing systems, communication enabled mobile telephones, personal digital assistants, or any other communication devices with which the patient or user of the device may use in conjunction therewith, in managing the treatment of a health condition, such as diabetes.

The system 100 may be configured to operate as an open loop system, a closed-loop system, and a hybrid closed-loop system. An open loop system requires manual user input to control certain functionalities related to the sensor assembly 300. A closed-loop system uses data from the sensor assembly 300 and algorithms to control the software library 400 without user input. In a hybrid system, input may be required from a user to control the application 420 and initiate the software library 400. A hybrid closed-loop system can be used in conjunction with, or in place of, a closed-loop system. As disclosed herein, regulatory clearance can be limited to software library 400 irrespective of the type of system configuration used in the system 100.

### Receiving Device

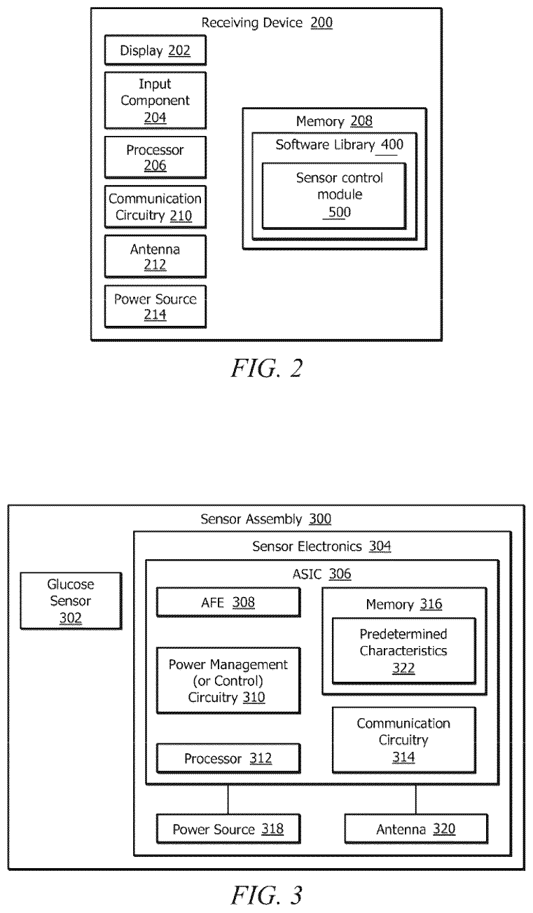

FIG. 2 is a block diagram depicting an example embodiment of a receiving device 200. A software library 400 can be provided to a third-party and incorporated within an application 420 for a multi-purpose receiving device 200, such as a mobile phone, tablet, personal receiving device, or other similar receiving device. Receiving device 200 embodying and executing device application software can also be referred to as a computing device or a multi-purpose device. Receiving device 200 refers to a suitably configured hardware device which is executing an application 420 that incorporates a software library 400 having a sensor control module 500 configured for communication with the sensor assembly 300. Here, receiving device 200 can include a display 202, input component 204, and a processor 206 coupled with memory 208. Also included can be communication circuitry 210 coupled with an antenna 212, and power source 214. As understood by one of skill in the art, these components are electrically and communicatively coupled in a manner to make a functional device. As embodied herein, the memory 208 can include an application and a sensor control module 500 for the sensor assembly 300. The application 420 can also import a software library 400 including the sensor control module 500. The software library 400 and the sensor control module 500 can be developed by the provider of the sensor assembly 300.

The receiving device can have the majority of the processing capability of the system 100 for rendering end-result data suitable for display to a user. The receiving device 200 can be a smartphone or a smartwatch.

The receiving device 200 can receive analyte data, such as glucose data and calculate low and high analyte level and generate corresponding alarms and messages. The receiving device 200 can also mirror an alert generated by another device, such as the sensor assembly 300. The receiving device 200 can process analyte data with the processor 206 and render on the display 202 analyte-related information as value, trend, and graph, and provide additional messaging and notification based on the received analyte level.

### Sensor Assembly

FIG. 3 is a block diagrams depicting an example embodiment of a sensor assembly 300 comprising a glucose sensor 302 and sensor electronics 304 (including analyte monitoring circuitry). Glucose sensor 302 can be an in vivo analyte sensor and have a use period of about 13-30 days. Sensor assembly 300 can be without wide-area network communication capability.

The glucose sensor 302 generates raw data signals for measurements of the patient's glucose level. Sensor electronics 304 are operatively coupled to the glucose sensor 302, the sensor electronics 304 comprising a memory 316 storing one or more predetermined characteristics 322 associated with the sensor electronics 304. The memory 316 can be a so-called “one-time programmable” (OTP) memory, which can include supporting architectures or otherwise be configured to define the number times to which a particular address or region of the memory can be written, which can be one time or more than one time up to the defined number of times after which the memory can be marked as unusable or otherwise made unavailable for programming. Subject matter disclosed herein relate to systems and method for updating said OTP memories with new information.

The sensor electronics 304 can include a single semiconductor chip, as depicted, that can be a custom application specific integrated circuit (ASIC 306). Shown within ASIC 306 are certain high-level functional units, including an analog front end (AFE 308), power management (or control) circuitry 310, processor 312, and communication circuitry 314 (which can be implemented as a transmitter, receiver, transceiver, passive circuit, or otherwise according to the communication protocol). As an example, only and not by way of limitation, example communication circuitry 314 can include a Bluetooth Low-Energy (“BLE”) chipset, Near-Field Communication (“NFC”) chipset, or other chipsets for use with similar short-range communication schemes, such as a personal area network according to IEEE 802.15 protocols, IEEE 802.11 protocols, infrared communications according to the Infrared Data Association standards (IrDA), etc. The communication circuitry 314 can transmit and receive data and commands via interaction with similarly capable communication modules. Certain communication chipsets can be embedded in ASIC 306 (e.g., an NFC antennae).

The sensor assembly 300 can use application layer encryption using one or more block ciphers to establish mutual authentication and encryption of other devices in the system 100. The use of a non-standard encryption design implemented in the application layer has several benefits. One benefit of this approach is that in certain embodiments the user can complete the pairing of the sensor assembly 300 and another device with minimal interaction, e.g., using only an NFC scan and without requiring additional input, such as entering a security pin or confirming pairing. Sensor assembly 300 can be configured to dynamically generate authentication and encryption keys. Sensor assembly 300 can also be pre-programmed with a set of valid authentication and encryption keys to use with particular classes of devices. The ASIC 306 can be further configured to perform authentication procedures with other devices (e.g., handshake, mutual authentication, etc.) using received data and apply the generated key to sensitive data prior to transmitting the sensitive data.

In this embodiment, both AFE 308 and processor 312 are used as analyte monitoring circuitry, but in other embodiments either circuit can perform the analyte monitoring function. Processor 312 can include one or more processors, microprocessors, controllers, and/or microcontrollers, each of which can be a discrete chip or distributed amongst (and a portion of) a number of different chips.

Memory 316 included within ASIC 306 and can be shared by the various functional units present within ASIC 306, or can be distributed amongst two or more of them. Memory 316 can also be a separate chip. Memory 316 can be volatile and/or non-volatile memory. In this embodiment, ASIC 306 is coupled with a power source 318, which can be a coin cell battery, or the like. AFE 308 interfaces with glucose sensor 302 and receives measurement data therefrom and outputs the data to processor 312 in digital form. This data can then be provided to communication circuitry 314 for sending, by way of antenna 320, to software library 400.

The glucose sensor 302 can alternatively monitor other analytes, for example, acetyl choline, amylase, bilirubin, cholesterol, chorionic gonadotropin, creatine kinase (e.g., CK-MB), creatine, DNA, fructosamine, glutamine, growth hormones, hormones, ketones, ketone bodies (e.g., β-hydroxybutyrate), lactate, peroxide, prostate-specific antigen, prothrombin, RNA, thyroid stimulating hormone, and troponin.

The sensor assembly 300 includes a sensor assembly embedded library (not pictured) configured for providing sensor assembly data to the software library 400 based on information received from the sensor assembly 300. Sensor assembly data can include glucose readings, data types, range, real time and historical glucose and trends, sensor operating information, and sensor system information.

### Software Library

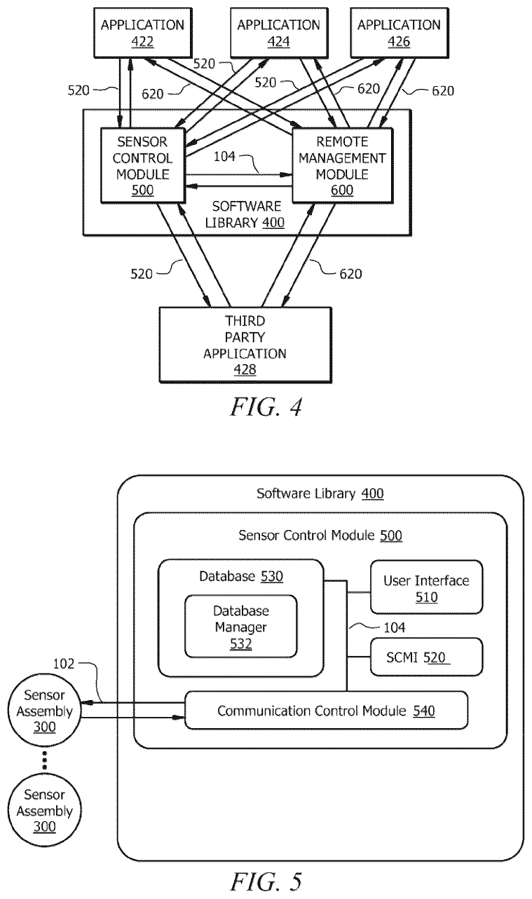

FIG. 4 is a block diagram depicting an example of a software library 400 for communication with applications 420, shown as applications 422, 424, 426, and third-party application 428. References to application 420 refers to one or more of the applications 422, 424, 426 or third-party application 428. Software library 400 includes a sensor control module 500 and a remote management module 600, each of which is capable of independently communicating with applications 422, 424, 426 or third-party application 428. In accordance with the disclosed subject matter, sensor control module 500 and a remote management module 600 may each provide a single uniform interface to communicate with the applications 422, 424, 426 or third-party application 428.

Software library 400 may use a modular architecture and may be made available via a software development kit that can be made for common use by applications 420. The software library 400 may include two modules, each of which could be independently provided for use by other applications 420. The first such module may be a sensor control module 500. The sensor control module may communicate with the sensor assembly 300 and receive a particular result of the value from the sensor assembly 300. The sensor control module 500 may further communicate with applications 422, 424, 426, or third-party application 428 using a sensor control module interface 520.

The software library 400 may further include a remote management module 600 that will be further described below. The remote management module 600 communicates with applications 422, 424, 426, or third-party application 428 using a remote management module interface 620.

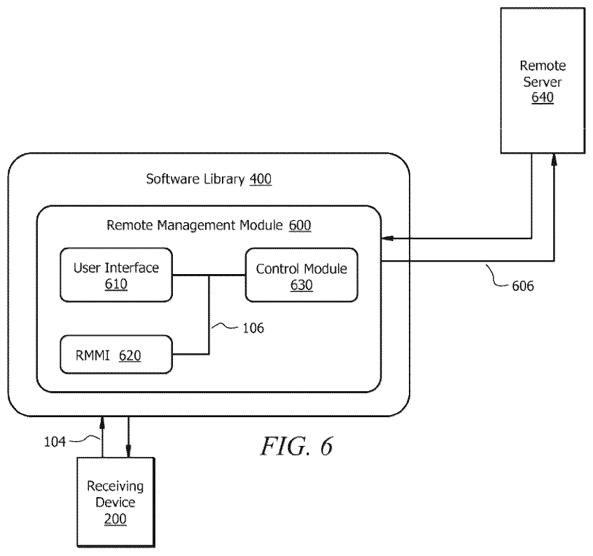

The remote management module 600 may further receive the sensor data from the sensor control module 500 via the inter-module interface 450 and can further be used to store that data in a remote server 640 (shown on FIG. 6) for remote storage, such as in the cloud. By using a remote management module 600, an application developer can also take advantage of a consistent user interface for account management for a user across different third-party applications such as third-party application 428. Data privacy can further be integrated into the remote management module 600 for account management purposes.

The sensor control module 500 may receive a request to initiate the sensor assembly 300. The sensor control module 500 may include logic to identify the particular type of receiving device 200 making the request, and can perform an authentication function for the receiving device 200. Authentication may use a three-pass design with different keys. Keys can be aligned with differential roles (manufacturer, application developer, etc.). Sensitive commands that could leak security information can trigger authenticated encryption using an authenticated additional keyset. The sensor data provided to the sensor control module 500 and sent to the application 422, 424, 426 or third-party application is highly sensitive and can be beneficial to be protected. Medical data associated with a patient is sensitive data at least in part because this information can be used for a variety of purposes, including for health monitoring and medication dosing decisions. As embodied herein, the various modules and applications 422, 424, 426, and third-party application 430 can be configured compliant with a security interface designed to protect the Confidentiality, Integrity and Availability (“CIA”) of this communication and associated data. To address these CIA concerns, to facilitate the confidentiality of data, communication connections between the sensor assembly 300 and the sensor control module 500 can be mutually authenticated prior to transmitting sensitive data. The same would be done for communication between the sensor control module 500 and application 422, 424, 426, and third-party application 428. Communication connections can be encrypted using a device-unique or session-unique encryption key. As embodied herein, the encryption parameters can be configured to change with every data block of the communication.

As embodied herein, to guarantee the integrity of data, encrypted communications between any two components (e.g., a sensor control module 500 and sensor assembly 300) can be verified with transmission integrity checks built into the communications. As embodied herein, session key information, which can be used to encrypt the communication, can be exchanged between two devices after the devices have each been authenticated. Encrypted communications between a sensor assembly 300 and a dedicated sensor control module 500 can be validated with an error detection code or error correction code, including as an example and not by way of limitation, non-secure error-detecting codes, minimum distance coding, repetition codes, parity bits, checksums, cyclic redundancy checks, cryptographic hash functions, error correction codes, and other suitable methods for detecting the presence of an error in a digital message.

The sensor control module 500 may further generate state information to maintain the active status for the receiving device 200 while it remains desirous of the sensor data.

The sensor control module 500 may include a user interface 510 that can enable data sharing for the applications, including necessary permissions to enable data sharing. The user interface 510 at the sensor control module 500 may also display the sensor data received from the sensor assembly 300.

The user interface 510 of the software library is disclosed herein as a modular user interface 510 that allows for sharing and display of the multiple different analytes that can be measured from the different sensor assemblies 300. In particular, as disclosed herein, by using a software library 400 and sensor control module 500, a shared user interface can be developed for display of sensor data from multiple sensor assemblies 300. The user interface 510, when shared, could toggle between sensor data related to the various sensor assemblies 300, display sensor data on one screen, or use multiple different combinations to display the sensor data.

Communication between the sensor control module 500 and applications 422, 424, 426, or third-party application 428 occurs over a sensor control module interface 520.

Communication between the remote management module 600 and applications 422, 424, 426, or third-party application 428 occurs over a remote management module 620. The communication is further driven using an event notification or callback process. For example, when the sensor control module 500 receives a request from a third-party application 428 for sensor data, the request may be communicated through the sensor control module interface 520 and an event may be generated at the user interface 510 of the sensor control module 500 to initiate authentication.

As another example, when sensor data is received over communication link 102 by the sensor control module 500, an event can be generated to notify other modules or components within the software architecture that data can be displayed on a user interface 510 of the sensor control module 500.

By having a modular architecture using a software library 400 and sensor control module 500 to interface with the applications 422, 424, 426 and third-party application 428, the system enables communication with different types of sensor assemblies 300, including multiple sensor assemblies 300. In particular, the communication control module 540 may include functionality specific to each of the sensor assemblies 300 within the system, and may simultaneously access and communicate with the various sensor assemblies 300 to receive sensor data.

As another example, a developer of a third-party application 428 may elect to use certain modules of the software library 400 to support the functionalities within the third-party application 428. For example, certain third-party applications 428 may use the sensor data as wellness data. Wellness data can generally include any type of data associated with a person's health, such as their weight, heart rate, blood pressure, blood glucose level, or the like. Sensor assemblies may provide resulting sensor data that may include such wellness data. To the extent a third-party application desires to make use of the sensor data, the third-party application may access the respective module from the software library 400 for the desired sensor data. With the software library 400, the third-party application 428 does not need to directly interface with the sensor assembly 300 to receive sensor data. The software library 400 includes a sensor control module 500 that can receive the sensor data and provide that to the respective third-party application 428. It should be understood that “third party” can correspond to an entity different than the manufacturer of the sensor assembly 300 or software library 400. The third-party application 428 may have access to certain permitted data on database 530 accessible through sensor control module interface 520. Separately, the third-party application 428 may include its own database (not pictured) for storing the sensor data received through the sensor control module 500.

In certain applications, software that operates in conjunction with a medical device such as a sensor assembly sensing data from a user interaction or user health information may be regulated as medical device software. Certain standards pertain to regulation of medical device software, including with reference to ISO 13485:2016 “Medical devices—Quality Management Systems—Requirements for regulatory purposes,” ISO 14971:2012 “Medical devices—Application of Risk Management to Medical Devices,” and IEC 62304, Ed 1.1:2015 Medical Device Software—Software Lifecycle Processes.” In particular, regulation requires that software that functions as a medical device (commonly referred to as Software as a Medical Device) is to be regulated by a regulatory agency, such as the Food and Drug Administration in the United States. This regulation at least requires submitting the application for regulatory clearance.

As described by the disclosed subject matter, the regulated portion of software as a medical device can be contained within the software library 400 and the sensor assembly 300. This can allow applications 422, 424, 426, or third-party application 428 not to have to undergo regulatory approval and clearance when making use of the sensor data. In particular, third-party applications may be developed by third-party developers for one or more wellness purposes that will not require the third-party developer to submit the application for approval based on definitions of software as a medical device as the regulated functionalities would all be contained within the software library 400. This will benefit users by allowing the creation of different wellness tracking applications or other uses of the sensor data that may not have originally been considered by the original manufacturer of the sensor assembly 300.

For applications 422, 424, 426, or third-party application 428, a sensor control module interface 520 is used to communicate with the sensor control module 500. By using the sensor control module interface 520, the applications 422, 424, 426 or third-party application 428 can receive data through the sensor control module 500.

The sensor control module 500 may optionally include an alarm module (not pictured) to manage alarms and notifications triggered by the sensor data. In accordance with the disclosed subject matter, the alarm module may include logic to generate alarms for each type of sensor measured by the sensor assembly 300. In particular, the alarms may be triggered if an issue arises with the device hardware of the sensor assembly 300. Additionally, the alarms may be triggered indicating a particular condition with the user being monitored by the sensor assembly 300. In accordance with the modular framework, the alarm logic for the alarm module may be separately maintained within the sensor control module 500.

As described herein, for illustration purposes, the alarm module works with the application 422, 424, 426 or third-party application 428 and the sensor control module 500. The sensor control module 500 receives sensor data from the sensor assembly 300 representing an analyte value. One such value could be a glucose reading. The sensor control module 500 and the alarm module may have threshold detection logic to identify the triggering conditions for an alarm based on a particular analyte value, such as a glucose reading.

During initialization, the third-party application 428 or application 422, 424, 426 can also provide conditions that would require the triggering of an alarm as a callback function. The triggering may involve logic that factors in the value of the sensor data and a temporal relationship. For example, if the sensor assembly provides glucose data, a triggering value may be set to trigger the alarm along with a temporal relationship such as if the value increases by a certain number over a period of time, or remains above a certain value for a period of time. These triggering conditions may also include rate of change as a mechanism to trigger an alarm. By incorporating the alarm module within the sensor control module 500, alarm conditions that require regulatory review and approval can be incorporated within the sensor control module 500, further reducing the need to submit application 422, 424, 426, or third-party application 428 for regulatory approval.

### Sensor Control Module

FIG. 5 is a block diagram depicting an example embodiment of the sensor control module 500 within a software library 400.

In certain embodiments, the sensor control module 500 includes a communication control module 540. The communication control module 540 includes logic to communicate over a communication link 102 to the sensor assembly 300. The communication control module 540 includes further logic for receiving sensor data and displaying the sensor data at a user interface 510. In particular, each sensor assembly 300 includes control logic to perform operations related to sensor communications, especially those that are proprietary. For example, the sensor assembly 300 includes logic provided by the sensor control device's manufacturer to receive sensor measurements and perform complex algorithms on the measurements including data decryption and glucose calculations. In this regard, the communication control module 540 may only need to receive the result of the processing and calculation, with data accuracy and integrity for protection of complex proprietary algorithms occurring at the closed sensor assembly 300. The sensor assembly 300 further includes logic provided by the sensor control device's manufacturer to perform authentication. This allows the sensor assembly 300 to include functionality to provide sensor data that is the resulting data from the sensor measurements for a variety of sensors to the communication control module 540. By using a modular framework, the communication control module 540 includes logic to receive data from a plurality of sensor assemblies 300, enabling substantially simultaneous communication from multiple sensor assemblies 300. This allows authorized third parties to develop mobile apps without requiring that those third parties take on the significant responsibility of independently providing the same level of performance and results accuracy.

This further enables various third-party companies to develop their own mobile applications that work with the manufacturer's sensor assemblies 300 through the software library 400 and the sensor control module 500, with those third-party companies having a variety of use cases that are different from those currently supported by the manufacturer. The utilization of a modular architecture enables third parties to only need to implement a smaller number of interface calls and reference the respective modular components of the software library 400 for implementation.

Communication within the sensor control module 500 to various components occurs over a sensor control module messaging channel 104. The user interface 510 can be used to display the sensor data once received over the sensor control module messaging channel 104.

Applications 422, 424, 426, or third-party application 428 include logic to communicate with the communication control module 540 over a sensor control module interface 520 and operate within the framework to enable receipt of sensor data. The application 420, 424, 426, or third-party application 428 requests sensor control module 500 to perform activation functions by first initiating the sensor control module 500 followed by sending a request to obtain sensor data. The sensor control module 500 includes a sensor control module interface 520 to ensure consistency for overlapping functions required of various applications 422, 424, 426 or third-party application 428. The sensor control module interface 520 is implemented as an application program interface (API) in the underlying application 422, 424, 426, or third-party application 428. A standard interface for shared functions also allows the sensor control module 500 to be used for receipt of sensor data from multiple sensors substantially simultaneously. Logic is contained within the software library for managing the activation of various applications 422, 424, 426 or third-party application 428 that have been authorized to receive sensor data. The sensor control module 500 may further include logic to control and manage the states of the various applications 422, 424, 426 or third-party application 428 via the sensor control module interface 520.

The sensor control module 500 within the software library 400 is positioned as a software as a medical device for regulatory clearance in conjunction with the sensor assembly 300. By housing the components that trigger software as a medical device regulatory issues within the software library communicating with the sensor assembly, additional third-party application 428 avoids the need to be submitted for regulatory approval. This further allows other application developers to build other use cases without having to submit the use-case for the application for regulatory review, and allows unregulated applications to take advantage of the sensor data. This advantage occurs by using the modular logic as described for the software library 400.

The user interface 510 provides a uniform interface for the applications 422, 424, 426 or third-party application 428 to display received sensor data. The user interface 510 may perform a user consent and onboarding function for the applications 422, 424, 426 or third-party application 428. Onboarding includes having a new user of the applications 422, 424, 426 or third-party application 428 completing the necessary consents to have access to the sensor data. The user interface 510 may further include a ready check to determine through the communication control module 540 that the various sensor assemblies 300 are functioning properly. The user interface 510 may include a display functionality to display the sensor data. The user interface 510 can be used in common form for the applications 422, 424, 426 or third-party application 428 for any number of shared functions, such as for account creation of a user, consents for data privacy and sharing, and other similar functions. In accordance with the disclosed embodiments, the sensor control module 500 may present a particular customized user interface 510 when application 422, 424, 426 developed by the manufacturer of the sensor assembly 300 is in operation, but a wholly different user interface 510 for a third-party application 428 that is not developed by the manufacturer of the sensor assembly 300. The look and feel of the user interface 510 thus automatically adjusts depending on whether the applications 422, 424, 426 or third-party application 428 has requested the sensor data. As disclosed herein, the sensor control module 500 may be implemented without a user interface 510 component. In this configuration, the sensor control module interface 520 functions to provide information directly to the display of the underlying applications 422, 424, 426 or third-party application 428.

The sensor control module 500 may optionally include integrity and initialization check of accounts to allow connectivity with the sensor and access to the sensor data. Applications 422, 424, 426 or third-party application 428 requests initialization of the sensor control module 500 on start-up of the applications 422, 424, 426 or third-party application 428 by supplying identifying information to the sensor control module 500 and credentials that the sensor control module 500 can use for authentication. If the integrity check fails, the sensor control module 500 will not allow for operation of that application 422, 424, 426 or third-party application 428. For third-party application 428, a remote management module 600 can be used to revoke access to the sensor control module 500 or remove authorization based on the manufacturer's current permissions and goals as determined by the connectivity between the remote management module 600 and the remote server 640. The remote management module 600 can also initiate a process to revoke the authentication of the third-party application 428 from the sensor control module 500 to prevent it from further operation. After successful initialization, the sensor control module 500 initializes the remote management module 600 by providing identifying information and credentials for authentication.

The sensor control module 500 may include protections to ensure that a proper authenticated application 422, 424, 426 or third-party application 428 had made requests for sensor data.

The communication control module 540 may communicate through the communication link 102 to the sensor assembly 300. Using a sensor control module messaging channel 104, the sensor data received from sensor assembly 300 is provided to other components of the sensor control module 500. The sensor data may also communicate to the remote management module 600 via another inter-module interface 450 between the sensor control assembly 500 and the remote management module 600. The sensor data may be further stored in database 530 managed by a database manager 532.

Because of the modular architecture of the software library 400, the communication control module 540 can receive data from any of the various types of sensors represented by sensor assembly 300. This allows for substantially simultaneous receipt of sensor data for the system. Support for multiple different types of sensors occurs at the system level in modular form allowing for future expansion as new sensors are built for tracking additional data by incorporating the necessary modules within the software library 400 and sensor control module 500.

The user interface 510 includes limited functionality to display the sensor data, such as glucose value, and is maintained in this form to allow for uniform use across multiple sensor readings for display of the sensor data. Processing and calculations occur at the sensor assembly 300, and the communication control module 540 receives that sensor data result as a value.

Once the communication control module 540 receives the sensor data, it may post an event by generating an event notification that will inform the respective application 422, 424, 426 that sensor data may be available and accessed through the sensor control module interface 520. The data may be stored in database 530 and accessed directly through the sensor control module interface 520. By using the sensor control module interface 520 and user interface 510, the sensor control module 500 presents a uniform interface for the various applications 422, 424, 426 or a third-party application 428 to activate and receive results of the sensor data. Additionally, the uniform interface 510 includes software logic to identify and register various applications 422, 424, 426 or third-party application 428 to receive certain types of sensor data via callbacks. As an example, if glucose sensor data is available, the uniform interface software logic through sensor control module interface 520 will invoke a callback within the applications 422, 424, 426, or third-party applications 428 authorized to receive glucose sensor data.

The uniform interface logic can use the unique identifier to identify the sensor assembly 300 for which the sensor data request is being made. Although not depicted, according to one aspect of the embodiments, a unique identifier object can be created as an initial step, if one does not already exist. In some embodiments, for example, the unique identifier object can be a user-specific identifier object (e.g., a username, a user profile, or a user account ID) that is inputted, generated, or facilitated by a software application, module, or routine within the software library 400 that is running on the application 420. In other embodiments, the unique identifier object can be associated with a physical device, e.g., a particular sensor assembly 300, and can comprise, for example, a serial number, a media access control (MAC) address, a public key, a private key, or a similar string of characters.

According to another aspect of the embodiments, each of the applications 422, 424, 426, or third-party application 428 includes parameters that can be passed to the sensor control module 500 when a respective call is made by an application 422, 424, 426, or third-party application 42. These various structures and data types can be made available to the sensor control module 500 to assist the sensor control module 500 in accessing the sensor assembly 300 to receive sensor data.

According to another aspect of the embodiments, the sensor control module 500 may store the meta data and state information associated with the sensor assembly 300 or application 422, 424, 426 or a third-party application 428. The sensor control module 500 may further store this data in encrypted form, such as by using the identifier related to the receiving device 200 or sensor assembly 300, state information, and any other information that is useful for establishing and maintaining a connection with the sensor assembly 300, application 422, 424, 426 or a third-party application 428. This database may be separate from the database accessible by the application 422, 424, 426 or a third-party application 428, despite being an active component (though generally inaccessible) component within the application 422, 424, 426 or a third-party application 428. An application 422, 424, 426 or third-party application 428 can also be deactivated or have its access removed from the sensor data.

The sensor control module 500 as embodied herein can identify the application 422, 424, 426 or third-party application 428 based on tag information. When a particular application 422, 424, 426 or third-party application 428 requests access to the sensor data, the sensor control module 500 may identify the application because the sensor control module 500 may be pre-loaded with tagging information corresponding to the application 422, 424, 426 or third-party application 428.

The current framework and system may be compatible with prior applications developed by the manufacturer of the sensor assembly 300. In particular, logic for converting sensor readings into usable data may be included within the sensor assembly 300 or within the respective application 422, 424, 426. In this manner, the system may take advantage of the framework to integrate prior developed applications into the framework of the system.

The sensor control module 500 also has logic to identify whether the request for sensor data comes from an application 422, 424, 426 or a third-party application 428. The sensor control module may further communicate information regarding a sensor data request to a remote management module 600.

The sensor control module 500 may also have logic to receive information regarding hardware issues with the sensor components of the sensor assembly 300. The sensor control module 500 may send a communication to the application 422, 424, 426 or a third-party application 428 to display a status message about an issue with the sensor assembly 300, such as by alerting the user through the application 422, 424, 426 or a third-party application 428 that a sensor is expiring, has a hardware malfunction, or some other problem that would interfere with providing sensor data related to the analyte being monitored by the sensor assembly 300. The sensor control module 500 may send a communication to the receiving device 200 operating system when the application 422, 424, 426 or third-party application 428 is in the background to display a notification identifying an issue with the sensor assembly 300. These issues may include that a sensor is expiring, has a hardware malfunction, or some other issue that would interfere with providing sensor data relating to the analyte being monitored by the respective sensor assembly 300.

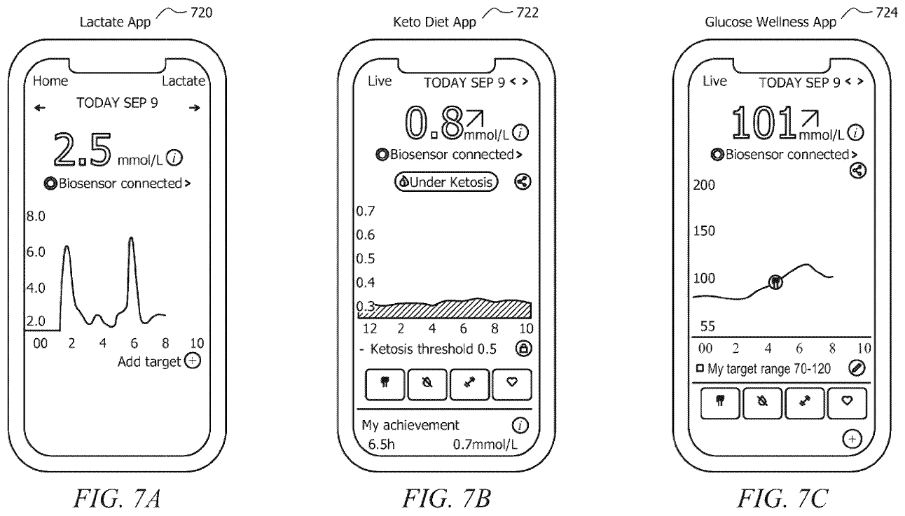

The application 422, 424, 426 or a third-party application 428 may include a user interface (shown further at FIGS. 7A-7C below), including a touch or voice command input, that acts as an interface to receive commands from a user. These commands or input may include a user requesting a sensor reading, visually tapping a display to get sensor data, acknowledging an alarm, or any number of different operations that could be conducted on the display of sensor data.

The sensor control module 500 may be coded in a modular fashion that allows for upgrading the software library 400 to add functionality to communicate with newly developed sensor assemblies. Variables are used in place of hard coded values to enable for modification of the sensor control module 500 to enable communication with newly developed sensor assemblies and to allow applications 422, 424, 426 or a third-party application 428 to get sensor data from those newly developed sensor assemblies without having to submit the underlying application in a new submission or an amended filing for regulatory review and clearance.

### Remote Management Module

FIG. 6 is a block diagram depicting an example embodiment of the remote management module 600.

The user interface 610 of the remote management module 600 provides functionality for applications 422, 424, 426 or a third-party application 428 to have a consistent interface for certain shared functions. As embodied herein, these features and functions can include activities such as data privacy, user consent, third-party consent, application authorization, and more. The user interface 610 of the remote management module 600 provides a consistent interface to allow various applications 422, 424, 426 or a third-party application 428 access to these functions. Communication within the remote management module 600 to various software logic can occur using the remote management module messaging channel 106. The user interface 610 also allows for consistent account management capabilities, allowing a user to create an account, set a password, or set profile related information.

The remote management module 600 further includes a remote control module 630 that enables communication to a remote server 640. The communication with the remote server 640 may occur wirelessly using any available communication means, including BLE and NFC communication.

In an embodiment of the system, the remote management module 600 may further provide transport capabilities for enabling a backup of data stored in the various applications 422, 424, 426 or a third-party application 428 in the event a user upgrades the smartphone or receiving device 200. The remote management module 600 may also communicate with the applications 422, 424, 426, or third-party application 428 over a remote management module interface 620.

The software library 400 including the sensor control module 500 and remote management module 600 may include secure coding layers to assist in the prevention of cyber threats, such hacking and remote access. In one example, protection against such threats may include the use of digital certificates or profile provisioning.

A sensor control module 500 can further identify whether the request for sensor data is generated by an application 422, 424, 426 or a third-party application 428. The sensor control module as embodied herein may pass that information to a remote management module 600 through inter-module interface 450, and the remote management module 600 can further customize the user interface 610 for that application 422, 424, 426 or a third-party application 428 using the remote infrastructure. As part of the customized user interface, a custom user authentication interface may be presented to a user of the application 422, 424, 426 or a third-party application 428. The remote management module 600 further includes logic to disable authentication for application 422, 424, 426 or a third-party application 428. In particular, allowing the remote management module 600 to disable access by a third-party application 428 by removing authorization for the third-party application 428 improves monitoring and control over the applications 422, 424, 426 or third-party application 428 that access sensor data.

### Set-Up

After initiation of set-up of the biosensor, a series of GUIs may be presented to assist the user in applying the biosensor to their skin surface and to pair the biosensor with the application. In some embodiments, the set-up GUIs may only be initiated by selecting the banner. In other embodiments, the set-up GUIs may be opened thorough a set-up button or settings link or button.

The application may present numerous GUIs that describe how to apply the biosensor. A GUI may be presented that shows what is included in the box. In addition to a picture, it may also explain that the user may find a biosensor pack and a biosensor applicator in the box. Another GUI may appear that instructs the user to choose a site on the back of their upper arm away from scars, moles, stretch marks, or lumps, and may be accompanied by a picture highlighting a section of a person's upper arm where it would be appropriate to apply the biosensor. An additional GUI may be provided that instructs the user to clean the selected site by washing the site using plain soap, cleaning the site with an alcohol wipe, and then allowing the site to dry.

The application may provide a GUI that explains how to prepare the biosensor pack and applicator. In addition to a picture showing how to open the pack, the GUI may contain written instructions to peel the lid completely off the biosensor pack and to unscrew the cap from the biosensor applicator. The application may then provide a GUI that includes a picture of a person loading the biosensor in the applicator along with instructions that state to line up the dark marks on the biosensor applicator and the biosensor pack. On a flat, hard surface, press down firmly on the biosensor applicator until it comes to a stop. The application may then provide another GUI that instructs the user to lift the biosensor applicator out of the biosensor pack and informs the user that the biosensor applicator is ready to apply the biosensor. The GUI may include a warning that the biosensor applicator now contains a needle and that the user should not touch inside the biosensor applicator or put it back into the biosensor pack.

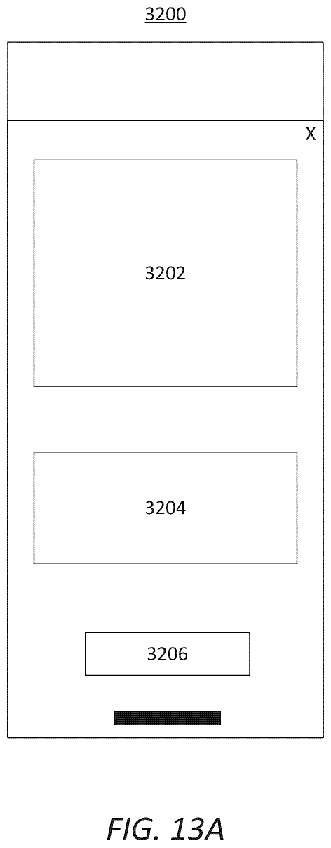

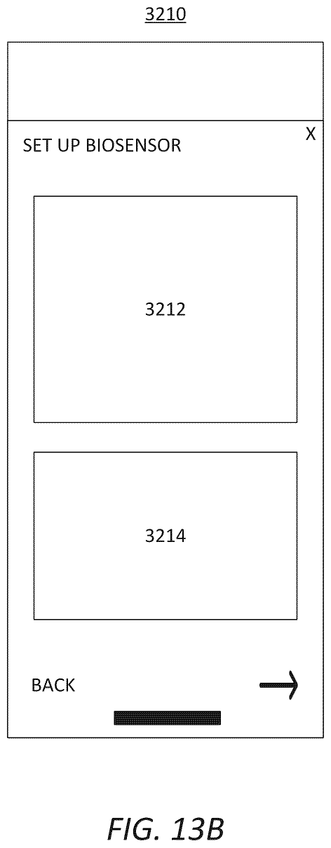

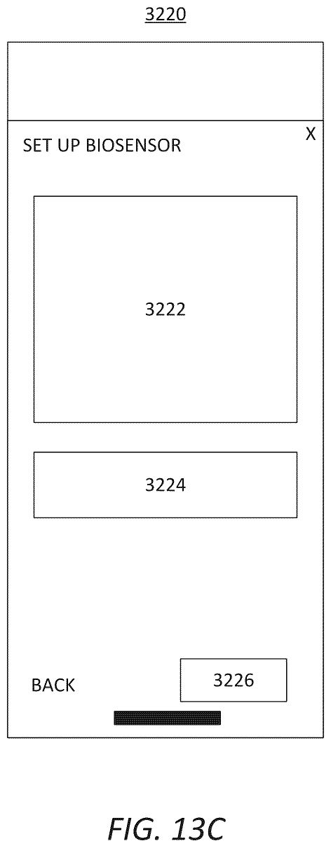

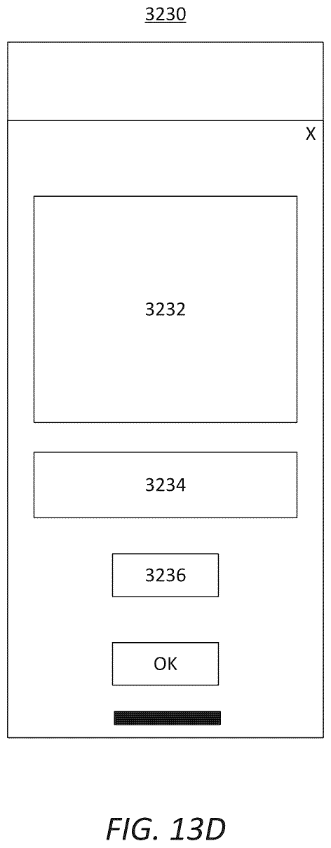

The application may then provide GUIs describing how to apply the biosensor to the user's body. As seen in GUI 3200 in FIG. 13A, an introductory GUI 3200 may include a graphic 3202 of a biosensor, along with introductory text 3204 inviting the user to begin the setup to pair their biosensor. The user may then tap or select a start setup button 3206 to begin the setup process.

As seen in GUI 3210 of FIG. 13B, a Set Up Biosensor GUI 3210 may include a graphic or picture 3212 showing a person applying an applicator to their body at, e.g., the back of their upper arm. The GUI 3210 may also include text 3214 explaining how to apply the biosensor. The text 3214 may instruct the user to place the biosensor applicator over a site and push down firmly to apply the biosensor. The text 3214 may then instruct the user to gently pull the biosensor applicator away from their body. The text 324 may also caution the user not to push down on the biosensor applicator until it is placed over the prepared site to prevent unintended results or injury. The user may then tap the arrow button to proceed to GUI 3220 after the biosensor has been applied.

The application may also present a GUI instructing the user to check the biosensor to make sure that the biosensor is secure by pressing down on the adhesive.

As seen in GUI 3220 of FIG. 13C, in another Set Up Biosensor GUI 3220, a graphic or picture 3222 may show a person pairing the biosensor to the reader device, such as a smart phone, by holding the reader device in close proximity to the applied biosensor. The text 3224 may instruct the user to pair their biosensor by tapping the start pairing button 3226. A pop-up window may appear that instructs the user to hold the reader device (e.g., mobile phone) very close to the biosensor. The phone may vibrate after successfully scanning the biosensor.

After the biosensor has been paired, as seen in FIG. 13D, GUI 3230 may be displayed, which indicates the time remaining for the biosensor to be ready. The GUI 3230 may include a graphic 3232 highlighting the time remaining until the biosensor is active. The graphic 3232 may include a radiating circle of dots, which may be animated. Alternatively, the graphic 3232 may include a progress indicator that may be a bar having a colored portion or may be a colored portion along the perimeter of a circle, where the colored portion is proportional to the amount of time remaining before the sensor is active, e.g., a total perimeter of a circle may be equivalent to 60 minutes and a colored portion of the perimeter may be proportional to the amount of time remaining under an hour for the biosensor to be active. Alternatively, the graphic 3232 may be animated and the color of the perimeter of the circle may change as the time remaining is counting down. The GUI 3230 may also include a display 3236 of the number of minutes until the biosensor is active (e.g., “55:10” where 55 minutes and 10 seconds remain until the sensor is ready or active). Alternatively, in some embodiments, a message of “Ready in 55 mins” may be displayed on the inside of a circle or below the graphic 3236, where a colored portion of the perimeter of the circle is animated and cyclically changes color as the time remaining until the biosensor is active counts down. The GUI may also include a plurality of selectable links that, when selected by the user, can display additional information to the user as explained elsewhere, including how to replace a biosensor, support, learning more about the application, and ordering a biosensor.

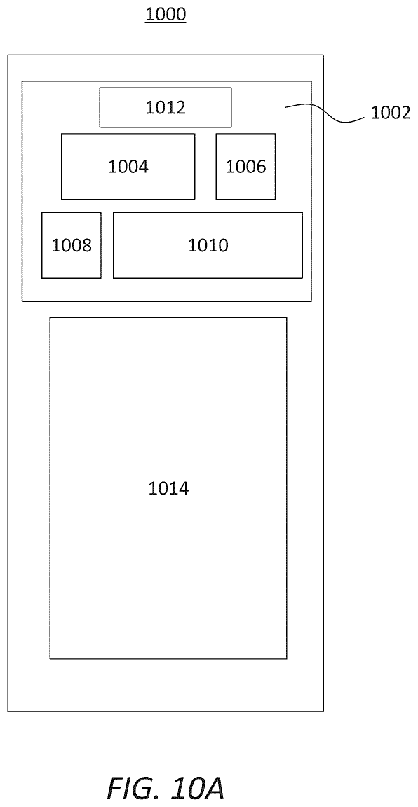

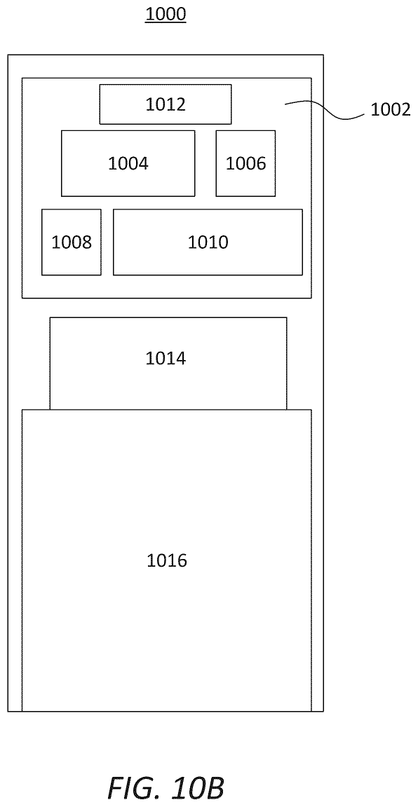

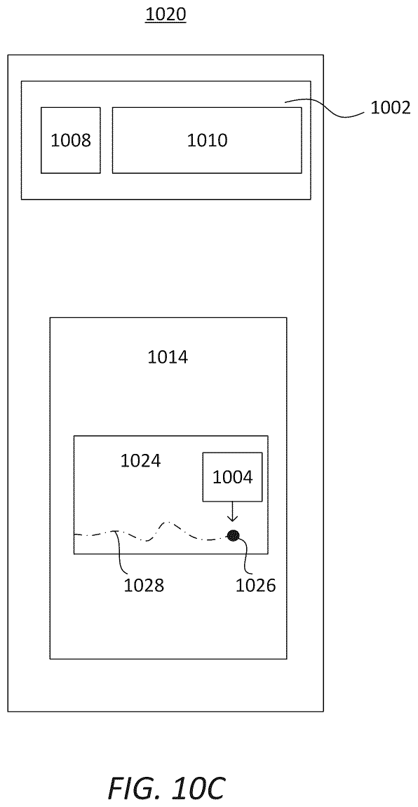

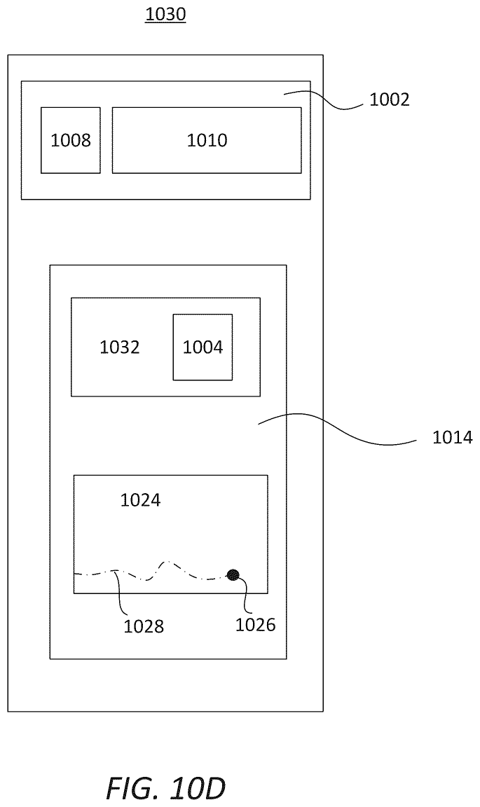

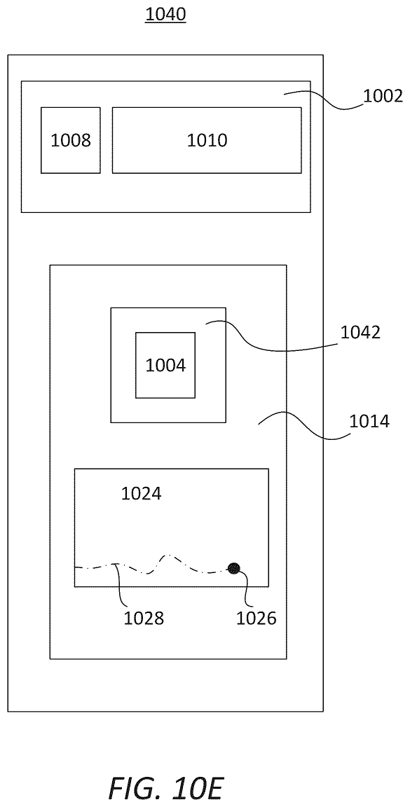

The GUI 3230 may also include a description or message 3234 that informs the user that their biosensor is now getting to know them, and real-time analyte levels will be available in the amount of time displayed in 3236. In some embodiments, elements of GUI 3230 may be provided by the biosensor module. For example, the graphic having a progress indicator 3232 and the display 3236 of the amount of time until the biosensor is active may be provided by the biosensor module. After the user clicks OK, the GUI 3230 may collapse and the home screen with the banner may then be visible to the user. As seen in FIG. 10A, the banner 1002 may display an icon 1008 indicating the status of the biosensor along with information 1010 regarding the status of the biosensor. In some embodiments, similar to the progress indicator described with respect to graphic 3232, the icon 1008 may also include a progress indicator that indicates the time remaining for the biosensor to be ready. The icon 1008 may include a graphic highlighting the time remaining until the biosensor is active. The graphic may include a radiating circle of dots, which may be animated. The icon 1008 may include a progress indicator that may be a bar having a colored portion or may be a colored portion along the perimeter of a circle, where the colored portion is proportional to the amount of time remaining before the sensor is active, e.g., a total perimeter of a circle may be equivalent to 60 minutes and a colored portion of the perimeter may be proportional to the amount of time remaining under an hour for the biosensor to be active. The information 1010 may include text indicating that the biosensor is “READY IN XX,” wherein XX may be displayed in minutes and seconds. For example, the banner 1002 may display “READY IN 55:10” where the biosensor will be active in 55 minutes and 10 seconds.

### Applications

FIG. 7A-7C are exemplary embodiments of applications using the software library 400 and sensor control module 500.

In one example, application 420 may be an application to track analyte values such as lactate as shown in FIG. 7A, ketones or ketone bodies (e.g., β-hydroxybutyrate), such as shown in FIG. 7B, or glucose, as shown in FIG. 7C. Part of the display may come from the sensor control module interface 520 and part may be displayed based on processing within the underlying application 420.

Furthermore, according to some embodiments, applications 720, 722, 724 represent applications 422, 424, 426 to communicate with the sensor control module 500 to enable receipt of sensor data. By using the sensor control module 500 and remote management module 600, a consistent user experience can be provided for the different applications. Moreover, if an additional analyte value needs to be detected and sensed, that application can further integrate the updated software library 400 without having to develop the full architecture for communication, account management, user privacy, and consents.

The improvements to the GUI in the various aspects described and claimed herein produce a technical effect at least in that they assist the user of the device to operate the device more accurately, more efficiently and more safely. It will be appreciated that the information that is provided to the user on the GUI, the order in which that information is provided and the clarity with which that information is structured can have a significant effect on the way the user interacts with the system and the way the system operates. The GUI therefore guides the user in the technical task of operating the system to take the necessary readings and/or obtain information accurately and efficiently.

### Biosensor Module Banner

As referenced above, user interface 510 of the sensor control module 500 may include various components. As seen in FIG. 10A, GUI 1000 may include a banner 1002, which is created by the user interface 510, that may be incorporated into GUIs generated by the host application, e.g., applications 422, 424, 426 or third-party application 428. The banner 1002 generated by the user interface 510 may show a different interface depending on the host application into which it is integrated. The banner 1002 may include a real time concentration value 1004, a trend arrow 1006, an icon 1008 indicating the status of the biosensor, information 1010 regarding the status of the biosensor, and an additional indication of the biosensor status 1012. The banner 1002, or elements within the banner 1002, may be selectable to link to other GUIs with additional information about the biosensor. For example, where the host application is a glucose wellness application, the banner 1002 may include a real time concentration value 1004, a status icon 1008, and information status 1010 about the biosensor. Moreover, the real time concentration value 1004 may be located in a different part of the GUI (e.g., as part of an analyte graph) than the rest of the banner.

Various different status icons 1008 and information regarding the status of the biosensor 1010 may be displayed. In some embodiments, a status icon 1008 including a circle of dots, which may or may not be animated, may appear next to status information 1010 indicating that the biosensor is not connected or ready. For example, the status information 1010 may state that the biosensor will be ready in a certain amount of time, e.g., “Ready in 30 mins.” In some embodiments, the status information 1010 may state “Searching,” which may indicate that the application is trying to connect to the biosensor.

In some embodiments, as seen in FIG. 10B, a pop-up screen 1016 may also appear over GUI 1000. The pop-up screen 1016 may convey a message regarding the biosensor starting up. The pop-up screen 1016 may indicate the amount of time in, e.g., hours and/or minutes, remaining until the biosensor is ready. For example, the pop-up screen may indicate that the “Biosensor is ready in 55 minutes.” In other embodiments, if the reader device, such as a smart phone, is locked, a notification may appear on the lock-screen indicating that the biosensor is ready.

As seen in FIG. 10A, in some embodiments, a status icon 1008 including a circle with a color progress indicator may appear next to status information 1010 stating that the biosensor is connected and working properly. For example, the status information 1010 may say “LIVE.” In some embodiments, for the status icon 1008 of the circle with the progress indicator, the progress indicator may be colored, and the color may be proportional to the amount of sensor life remaining for the current biosensor. In some embodiments, the color of the progress indicator may be different colors depending on how much sensor life is remaining. For example, the color indicator may be a blue color if at least about 50%, alternatively at least about 40%, alternatively at least about 30%, alternatively at least about 25%, alternatively at least about 20%, alternatively at least about 10% of the sensor life remains. In some embodiments, the colored progress indicator may be a different color, e.g., orange or red, if the sensor life remaining is less than a certain amount. For example, the color indicator may be an orange color if less than about 50%, alternatively less than about 40%, alternatively less than about 30%, alternatively less than about 20%, alternatively less than about 10%, alternatively less than about 5% of the sensor life remaining.

In some embodiments, the status information 1010 may state “SEE DETAILS” or similar language to indicate that the user should learn more about the status of the biosensor. By selecting on the “SEE DETAILS” or other parts of the banner, one of many explanations, may be displayed to indicate a possible error or issue with the biosensor. In some embodiments, where there is an issue with the biosensor and the status information 1010 states “SEE DETAILS,” a real time concentration value 1004 may not be displayed. In lieu of the real time concentration value 1004, in some embodiments, a plurality of dashes or dots (e.g., two dashes) may appear instead of the concentration value 1004 of the analyte measured by the biosensor.

In some embodiments, the banner 1002 may include a real time concentration value 1004, an icon 1008 indicating the status of the biosensor, and information 1010 regarding the status of the biosensor. As seen in FIG. 10C, a GUI 1020 may have the real time analyte concentration value 1004 located in a different part of the GUI than the other components of the banner 1002. For example, the real time analyte concentration value 1004 may be located in a graph 1024 that is part 1014 of the GUI generated by the host application. The graph may include analyte curve 1028 and a marker 1026 for the current analyte concentration, and the real time analyte concentration value 1004 may be located above the marker 1026. In other embodiments, as seen in FIG. 10D, the real time analyte concentration value 1004 in GUI 1030 may be located in a sentence or statement 1032 about the user's analyte concentration. For instance, the sentence or statement 1032 may be located above the graph 1024 and may say, for example, “Your glucose is 124 mg/dL. Your number is steady, and you're doing great!”

In some embodiments, the banner 1002 may include a real time concentration value 1004, an icon 1008 indicating the status of the biosensor, and information 1010 regarding the status of the biosensor. As seen in FIG. 10E, the real time analyte concentration value 1004 in GUI 1040 may be located in a graphic element 1042 provided by the host application. The graphic element 1042 may be a colored circle or other shape. If the analyte level is within a target range or determined to be steady, as explained elsewhere in this application, the graphic element 1042 may be colored a first color (e.g., green). If the analyte level is outside of a target range, determined to be unsteady (as explained elsewhere in this application), or a spike in the analyte concentration is detected, the graphic element 1042 may be colored a second color (e.g., orange). The color of the graphic element may be the same color as the analyte curve 1028 in the analyte graph 1024.

### System Messages

If there is an issue with the biosensor, a system message may appear regarding the status of the biosensor. The system message may appear in a pop-up window or alert. Alternatively, in some embodiments, the system message may appear after the user selects “SEE DETAILS.”

In some embodiments, the details message 1210 may include a “Pairing Error” message that may state that the pairing was unsuccessful. Moreover, the application may suggest trying to pair the biosensor again.

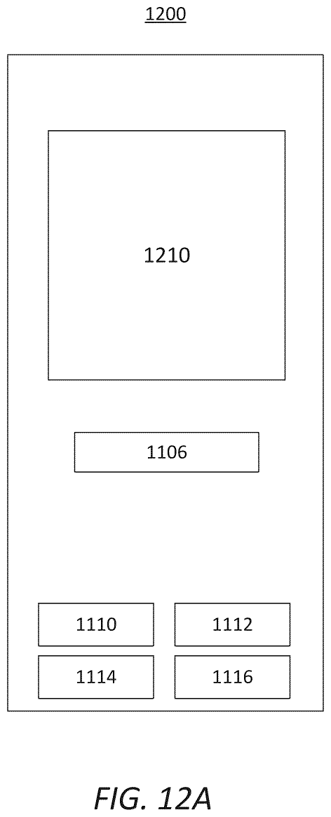

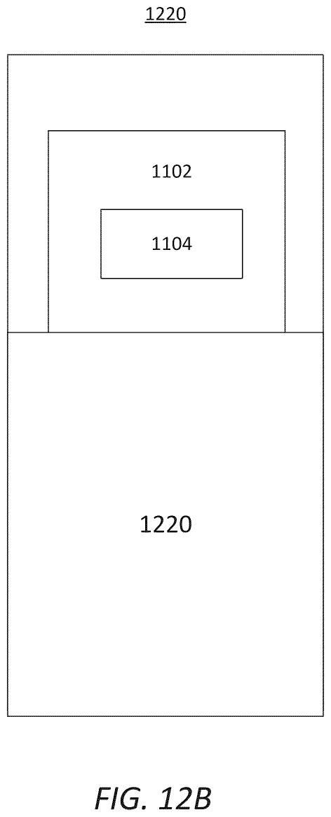

After a user selects “SEE DETAILS,” as seen in FIG. 12A, user interface 510 of the sensor control module 500 can be configured to display one of a plurality of messages in a GUI 1200 that may include a details message 1210, the serial number of the current biosensor 1106, and a plurality of selectable links 1110, 1112, 1114, 1116 that, when selected by the user, can display additional information to the user as explained elsewhere. In some embodiments, the details message 1210 may be presented in a circular graphic that includes a progress indicator to visually illustrate the remaining life of the current biosensor. As explained with respect to other embodiments, the graphic may be a circle and the progress indicator may be a different color perimeter of the circle, where the progress indicator may be proportional to the amount of sensor life remaining for the current biosensor. And as explained with respect to other embodiments, the color of the progress indicator may be different colors depending on how much sensor life is remaining.

In some embodiments, the banner 1002 may display a plurality of dashes or dots (e.g., two dashes) instead of the concentration value or level 1004 of the analyte measured by the biosensor when there is an issue with the biosensor. If the user taps on the banner 1002 when the banner is not displaying a real-time analyte concentration or level, e.g., displaying plurality of dashes or dots, then one of a plurality of system messages regarding problems with the biosensor may be displayed. When the user taps or selects the banner, a details message GUI may appear that gives additional details about the biosensor status.

In some embodiments, the details message 1210 may include a “Check Biosensor” message that may state that it looks like the user's biosensor isn't applied correctly. The details message 1210 may also include further instructions indicating if the biosensor is not attached firmly to the user's skin, then the user should apply a new biosensor and pair it. The details message 1210 may also include further instructions indicating if the biosensor is applied properly, then the user should try pairing the biosensor again. The details message 1210 may optionally also include a selectable “Pair” or “Pair Biosensor” button that, when selected, would display a GUI that assists the user to begin the pairing process.

In some embodiments, the details message 1210 may include a “Signal Loss” message that may caution the user to keep their phone in range of the biosensor at all times. The details message 1210 may further instruct that if the user continues having issues, then they should turn their phone's Bluetooth off and on, or restart their phone.

In some embodiments, the details message may relate to the temperature of the biosensor. In some embodiments, the details message 1210 may include a “Biosensor Too Hot” message that may inform the user that the biosensor is too hot to give readings. The details message 1210 may further request that the user to please check again in a few minutes. In some embodiments, the details message 1210 may include a “Biosensor Too Cold” message that may inform the user that the biosensor is too cold to give readings. The details message 1210 may further request that the user to please check again in a few minutes.

In some embodiments, the details message 1210 may include a “Biosensor Error” message that may inform the user that a biosensor reading is unavailable. The details message 1210 may further request that the user to please check again in a certain time period, e.g., 5 minutes, alternatively 10 minutes, alternatively 30 minutes.

In some embodiments, when the signal from the biosensor is suddenly lost, the biosensor status may change immediately to “SEARCHING”. When the “searching” description is selected or tapped, a details message 1210 may appear that suggests that the user keep their phone in range of the biosensor at all times. If the user continues having issues, the application suggests that the user turn the phone's BLUETOOTH® off and on, or restart the phone.

In some embodiments, the details message 1210 may include a “Biosensor Incompatible” message that may inform the user that the biosensor cannot be used with this version of the application. The message may suggest removing the biosensor and pairing a new one.

In some embodiments, the details message 1210 may include a “Biosensor Ended” message that may inform the user that the biosensor has ended and instruct the user to pair a new biosensor.

In some embodiments, the details message 1210 may include a “Biosensor already in use” message that may inform the user that a biosensor has already been paired and cannot be used. The message may also instruct the user to remove the biosensor and pair a new one.

In some embodiments, the details message 1210 may include a “Current Biosensor” message, which may be in a different color than messages indicating problems or errors. The message may indicate that the biosensor that the user tried to pair is in use and that the user will automatically receive real-time analyte readings directly to their device. The message may also include a different icon, such as a check mark.

In some embodiments, the details message 1210 may include an “ENABLE BLUETOOTH” message that requests the user to please turn on BLUETOOTH. The details message 1210 may further explain that BLUETOOTH is required to receive biosensor readings.

In some embodiments, the details message 1210 may include a “Replace Biosensor” message that may inform the user that the biosensor is not working. The details message 1210 may further request that the user please remove the biosensor and pair a new one.

As seen in FIG. 12B, a pop-up window 1220 may also appear, which may include a details message. The contents of the details message in pop-up window 1220 may be the same or substantially the same as described with respect to the plurality of details message 1210 described above that my appear in GUI 1200.

The biosensor module may also create and store an error log.

### Biosensor Module Details GUI

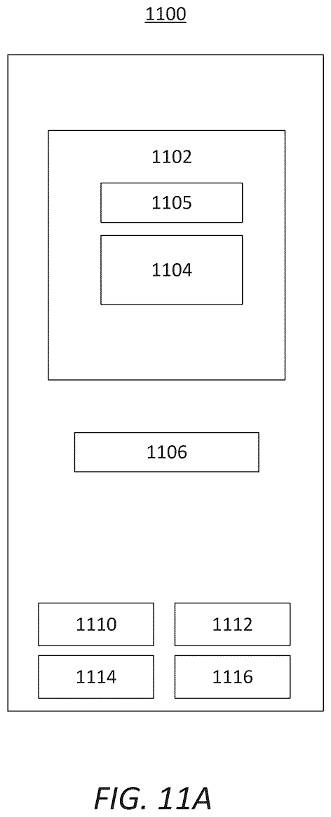

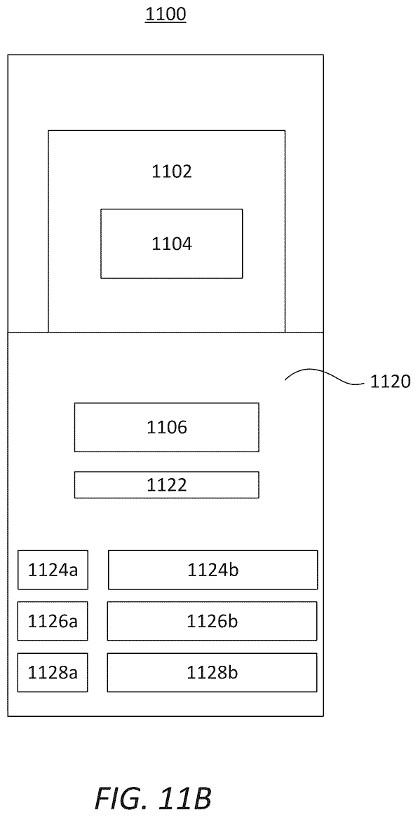

If the user selects any of the elements of the banner 1002, user interface 510 of sensor control module 500 can be configured to output a biosensor module details GUI 1100, as seen in FIG. 11A. The biosensor module details GUI 1100 may include a graphic 1102 that includes a progress indicator to visually illustrate the remaining life of the current biosensor. In some embodiments, the graphic 1102 may be a circle and the progress indicator may be a different color perimeter of the circle, where the progress indicator may be proportional to the amount of sensor life remaining for the current biosensor. For example, for a biosensor with a total life of X days, where the biosensor has Y days of life remaining, then the color indicator around the circumference will be a different color, e.g., blue, for Y/X*100 of the circumference of the circular graphic 1102. For example, for a biosensor with a total life of 14 days, where the biosensor has 12 days of life remaining, then the color indicator around the circumference will be blue for approx. 85.7% of the circumference of the circle 1102.

In some embodiments, the color of the progress indicator may be different colors depending on how much sensor life is remaining. For example, the color indicator may be a blue color if at least about 50%, alternatively at least about 40%, alternatively at least about 30%, alternatively at least about 25%, alternatively at least about 20%, alternatively at least about 10% of the sensor life remains. In some embodiments, the colored progress indicator may be a different color, e.g., orange or red, if the sensor life remaining is less than a certain amount. For example, the color indicator may be an orange color if less than about 50%, alternatively less than about 40%, alternatively less than about 30%, alternatively less than about 20%, alternatively less than about 10%, alternatively less than about 5% of the sensor life remaining.

The GUI 1100 may also include a real time indicator of amount of time remaining of the biosensor life 1104. For instance, the real time indicator may state “12 days” in the middle of the circular graphic 1102, in the example above. The real time indicator 1104 may be proportional to the progress indicator of the graphic 1102. The real time indicator 1104 may indicate the amount of time remaining of the biosensor life as a number of days when the amount of time remaining is greater than about 1 day, alternatively greater than about 23 hours and 59 minutes. The real time indicator 1104 may indicate the amount of time remaining as a number of hours when the amount of time remaining of the biosensor life is less than about 24 hours. In some embodiments, when the amount of time remaining of the biosensor life is less than about 24 hours, then the progress indicator may switch to a different color, e.g., the color may change from blue or green or orange or red. The real time indicator 1104 may indicate the amount of time remaining as a number of minutes when the amount of time remaining of the biosensor life is less than about 1 hour or less than about 61 minutes. The GUI may also include an additional message 1105 stating that the Biosensor Life is “Ending Soon.”

The GUI 1100 may also list the serial number of the current biosensor 1106, which may assist the user if seeking assistance with customer service. If the user selects the serial number 1106, as seen in FIG. 11B, a pop-up screen 1120 with additional details about the biosensor may appear. The pop-up screen 1120 may include the serial number of the current biosensor 1106 and the status of the current biosensor 1122. The pop-up screen 1120 may also include a list of past biosensors 1124, 1126, 1128. The list of past biosensors may include a date 1124a, 1126a, 1128a associated with each biosensor (e.g., the date of activation, or the date that the biosensor was disconnected), along with the serial number and status of the past biosensor 1124b, 1126b, 1128b. In some embodiments, the serial numbers of the current and past biosensors and other information may be copied to assist the user in conveying this information if needed to, e.g., their HCP or customer support.

The GUI 1100 may also include a plurality of selectable links 1110, 1112, 1114, 1116 that, when selected by the user, can display additional information about how to apply the biosensor, options to buy additional biosensors, instructions for use, how to replace a biosensor, support, learning more about the application, learning more about the biosensor, etc.

### Support

If the user taps or selects the link to “Support,” a GUI may include a Help and Learning section, an ABOUT section, and a Customer Care section, which includes details about how to contact a help line. The Help and Learning section may include links to FAQs, Understanding Glucose Readings, and App Tutorial. The ABOUT section may include links to Biosensor IDs, Error History, and App and Biosensor information. As explained elsewhere, the Biosensor IDs link may display a GUI that includes the serial number of the current biosensor, along with a list of past biosensors. The list of past biosensors may include a date associated with each biosensor (e.g., the date of activation, or the date that the biosensor was disconnected), along with the serial number and status of the past biosensor. The Error History link may display a GUI listing each error code, a brief description, and a time and date that the error occurred. The App and Biosensor information link may display a GUI listing the name of the application, the software full version, the SDK version, the OS version, the smartphone model, the country, and a reference number.

### Notifications

In some embodiments, the biosensor module may provide in-app notifications to alert the user of the status of the biosensor. The in-app notifications may also alert the user as to possible actions to take related to the biosensor. The biosensor module may function to facilitate the host application to display operating system-based notifications related to the status of the biosensor (e.g., informing the user that the biosensor has ended). The in-app notifications may appear in a pop-up notification with the host application in the foreground. In some embodiments, the in-app notifications provided by the third-party application related to the status of the biosensor may only be provided by the biosensor module.

The notifications may relate to the status of the biosensor, as described elsewhere herein. The notifications may relate to enabling BLUETOOTH® and alert the user that Bluetooth is required to receive Biosensor readings, and the notification may request that the user turn Bluetooth on now. The notifications may relate to checking their biosensor and alert the user that it looks like their Biosensor is not applied properly. The notifications may relate to replacing the Biosensor, and alert the user that their Biosensor is not working, instructing the user to remove the Biosensor and pair a new one. The notifications may relate to the end of a Biosensor session, alerting the user that their Biosensor session is completed and that it is time to pair a new Biosensor and continue to learn about their glucose levels. The notifications may relate to the Biosensor being ready, and state that Biosensor data is being received and will be displayed automatically. The notification may also invite the user to explore the glucose wellness application. The notifications may relate to the Biosensor ending in a certain number of hours, and request that the user buy a new Biosensor and replace the current Biosensor soon. The number of hours remaining may be 24 hours, 12 hours, 6 hours, 1 hour, or 30 minutes. The notifications may relate to signal from the Biosensor being lost and indicate that the user's phone is out of range of the Biosensor. The notifications may relate to the Biosensor being too hot to give readings, and request that the user check again in a few minutes. The notifications may relate to the Biosensor being too cold to give readings, and request that the user check again in a few minutes. The notifications may relate to a Biosensor error and inform the user that Biosensor reading is unavailable, and request that the user check again in 10 minutes.

### Biosensor Ended/Session Completed

When a biosensor session has ended and it is time to replace the biosensor, the sensor control module 500 may cause a reminder to pair a new biosensor in numerous ways. In some embodiments, as seen in FIGS. 10A-10B, an icon 1008 and a status information message 1010 may appear that reminds the user to start a new biosensor. The icon 1008 for starting a new biosensor may be a different icon than others used regarding the status of the biosensor and may resemble a full moon, a light bulb, etc.

In some embodiments, a pop-up window 1016 may appear, either in addition to the status information message 1010 or in the alternative, and remind the user that the biosensor session has ended and a new biosensor must be paired. In some embodiments, if the life of the biosensor is 14 days, a pop-up winder 1016 may appear when the user has just finished a 14-day session and may say that the biosensor session is completed. The pop-up window 1016 may also remind the user that it is time to replace the biosensor by pairing a new biosensor. The pop-up window 1016 may include also include a selectable “Pair” or “Pair Biosensor” button that, when selected, would display a GUI that assists the user to begin the pairing process. The pop-up window 1016 may also include a link to instructions regarding how to apply the biosensor and/or a link to a website where the user may purchase another biosensor.

In some embodiments, as seen in FIG. 10B, the pop-up window 1016 may appear when the user comes back to the host application but has not set up a new sensor. In some embodiments, the pop-up window 1016 may welcome the user back to the application. The pop-up window 1016 may also remind the user to pair a new biosensor. The pop-up window 1016 may include also include a selectable “Pair” or “Pair Biosensor” button that, when selected, would display a GUI that assists the user to begin the pairing process. The pop-up window 1016 may also include a link to instructions regarding how to apply the biosensor and/or a link to a website where the user may purchase another biosensor. When the user is not browsing the application or when the application is in the background or closed, notifications may appear on the device, e.g., on the lock screen. In some embodiments, the notification may indicate that “Your session is completed!” and suggest that the user pair a new biosensor to keep learning about their body.

The biosensor module details GUI 1100 may also display a reminder to start a new biosensor in as a message associated with graphic 1102, as seen in FIGS. 11A-11B. The message may remind the user to start a new biosensor. The message may also advise the user to please remove the biosensor and pair a new biosensor. GUI 1100 may also include a selectable “Pair” or “Pair Biosensor” button that, when selected, would display a GUI that assists the user to begin the pairing process. In some embodiments, the pop-up window 1120 may appear with a reminder that the new biosensor is ready to scan after the user selects the “Pair” or “Pair Biosensor” button. The pop-up window 1120 may include a graphic of a phone or reader device and may also include instructions reminding the user to hold the top of the phone very close to the biosensor. The pop-up window 1120 may also remind the user that the phone will vibrate or otherwise notify the user (e.g., a sound) after successfully scanning the biosensor.

As mentioned with respect to different GUIs, e.g., the Details GUI, the application may provide a link to “how to replace your biosensor,” which may be helpful to new users. If the user selects the link to “how to replace your biosensor,” a GUI may be presented with selectable options, (1) removing the biosensor, and (2) applying a new biosensor. The GUI may also include an option for replacing the current biosensor before it ends.

If the user wants to remove the biosensor, a GUI may be displayed that includes a warning that the action of removing the biosensor is not reversible. Once the user removes their biosensor, they will need to start a new one. The GUI may also contain instructions to pull up the edge of the adhesive that keeps the biosensor attached to the user's skin and slowly peel away the adhesive from their skin in one motion. If any remaining residue remains on the skin, it can be removed with warm soapy water or isopropyl alcohol. The GUI may also contain instructions for the user to discard the used biosensor according to their local regulations. Furthermore, it may instruct the user that when they are ready to apply a new biosensor, follow the instructions in a provided link to “Apply New Biosensor.” By selecting or tapping the link to “Apply New Biosensor,” the GUIs described in the set-up section may appear.

If the user opts to replace the biosensor before it ends, a pop-up window may appear asking if the user wants to end the biosensor early. If the user taps “END BIOSENSOR,” this will force-end the current biosensor and the user will need to remove it and apply a new biosensor. The pop-up may also contain a warning that this action is not reversible. If the user selects the link to end the biosensor, then the application may display the GUIs associated with replacing the biosensor discussed elsewhere.

After the first biosensor has ended, a pop up may appear that prompts the user to rate the monitoring application.

### Replace Biosensor due to Sensor Errors

The application may display a pop-up or alert if the biosensor needs to be replaced. The pop-up or alert may include a warning icon (e.g., an orange triangle with an exclamation point) that warns the user that their biosensor is not working and instructs the user to please replace their biosensor and pair the new biosensor. The pop-up may also include selectable links to “pair” or “pair biosensor” or instructions on how to replace their biosensor. The live screen may also include indications that the biosensor is not working properly. Instead of displaying a current analyte level, the banner may display dash marks (e.g., “--”) instead of a numerical value, and the informational text may state “SEE DETAILS.” If the user taps on any part of the banner, the Details GUI, described elsewhere, may appear with the message to replace their biosensor. As described elsewhere, the Details GUI may include links to additional information. For example, the Details GUI may include a link to how to replace the biosensor, order a biosensor, support, and information about the monitoring application.

Communication between Sensor and Applications

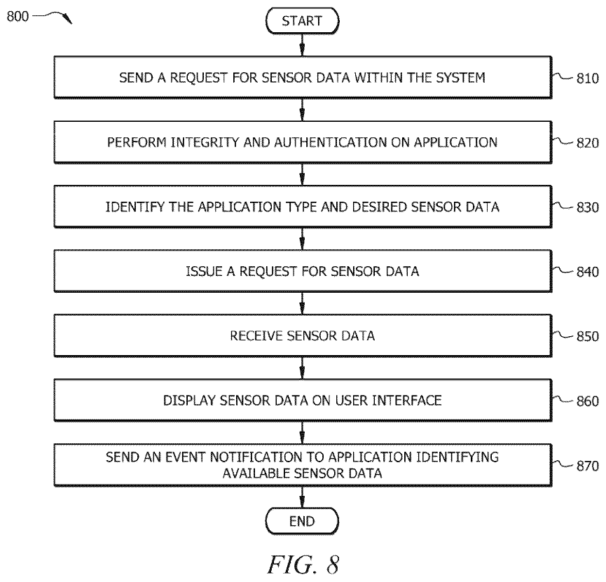

FIG. 8 depicts an example method for communicating sensor data from a sensor to a third-party application 428. As an initial matter, it will be understood by those in the art that any or all of the method steps and/or routines described herein may comprise instructions (e.g., software, firmware, etc.) stored in non-volatile memory of a sensor control device, a remote device (e.g., smartphone, reader), and/or any other computing device that is part of, or in communication with, an analyte monitoring system. Furthermore, the instructions, when executed by the one or more processors of their respective computing device, can cause the one or more processors to perform any one or more of the method steps described herein. Computing device may be the receiving device 200. In addition, although one or more of the method steps and/or routines described herein may comprise software and/or firmware stored on a single computing device, those of skill in the art will recognize that, in certain embodiments, the software and/or firmware may be distributed across multiple similar or disparate computing devices or software modules.

According to one aspect of the embodiments, method 800 can support an application 422, 424, 426 or a third-party application 428 from receiving sensor data for use within the application. At step 810, a third-party application 428 sends a request for sensor data within the system. The request routes to sensor control module 500 through the sensor control module interface 520; the second control module 500 communicates to the sensor assembly 300 using communication control module 540. At step 820, the sensor control module 500 verifies the authenticity of the third-party application 428 and integrity of the session. The sensor control module 500 may further communicate with the remote management module 600 to support user authentication and obtain content specific information for the third-party application 428. These modules may be available within a software library 400 so that a third-party application 428 developer can integrate as a framework within the system of the third-party application 428. At step 830, the sensor control module 500 using logic can identify the third-party application type and desired sensor data.

At step 840, the sensor control module 500 can issue a request for sensor data to the sensor assembly. Alternatively, the sensor control module 500 can be in receipt of sensor data based on a predetermined transmission rate (e.g., every 30 seconds, every minute, every 5 minutes, etc.). According to some embodiments, the sensor data can comprise data indicative of an analyte level, such as, for example, a glucose level, a glucose rate-of-change, a glucose trend, or a glucose alarm condition, among others. At step 850, the sensor data is delivered through a communications link 102 and stored within the database 530 of the sensor control module 500, and displayed at the user interface 510 as shown at step 860.

The sensor control module 500 database 530 includes and can store sensor data separately for each value generated by the various sensor assemblies 300. Database manager 532 may control one or more databases 530 with each separately storing the different types of sensors comprising the sensor assemblies 300. The data may also be stored together within a single database 530. The pictured database 530 is for illustration purposes, not limitation. Separate databases may also be dedicated to storing alarm conditions and triggered alarm results or notifications for each alarm at the sensor control module 500 database 530.

The user interface 510 may also be used to generate alarm notifications to users for alarms that have been triggered based on the sensor data or based on the condition of the sensor assembly 300. The sensor control module 500 may need to alert a user concerning the presence of an alarm. That communication would occur through the sensor control module interface 520 and driven by the user interface 510.

The disclosed subject matter further includes that the remote management module 600 may store alarm notifications and events for the application 422, 424, 426, or third-party application 428 as a backup at the remote server 640. This would allow alarm events to be generated for application 422, 424, 426 or a third-party application 428 that can be stored outside of a module that requires regulatory review and approval. In this manner, different applications developed to monitor a user's health and wellness can use the alarm events for wellness purposes that do not require regulatory clearance. The application 422, 424, 426 or a third-party application 428 may also store sensor data, alarm conditions, or notifications within its own database or shared database separate from database 530 within the sensor control module 500.

As disclosed herein, one such benefit of the software library 400 and modular approach of using the sensor control module 500 is that it would allow users and application developers to identify and develop different wellness related applications for the sensor data. This would enable users that do not traditionally use tracking of analytes, such as glucose monitoring, to consider adding it for purposes of health and wellness such as food tracking, customizable diabetes management, and other unregulated uses. By having the modular sensor control module 500, third-party applications 428 could use the sensor data in any unregulated manner without having to perform the regulatory clearance process. This in turn would expand the user-base for a manufacturer's sensor assemblies 300 by virtue of having more functions available for a user considering using the manufacturer's sensors. Those features can be implemented and improved on these third-party applications 428 without having to submit the revised improvements for regulatory review and clearance, further demonstrating how this disclosure improves initiatives to target wellness for users.

This allows the software library to be expandable to use the sensor control module 500 to collect and provide sensor data for yet to be developed sensors. The modular approach disclosed herein would reduce the need to rewrite code for shared functions and approaches to reading data from the various existing sensors and newly developed sensors, minimizes costs for introducing new sensors, and increases the functions and options for use of that sensor data in a wellness application. The expandable configuration allows the overall system to be extendable to future generations of sensor assemblies 300 and applications of the sensor data to additionally promote wellness use cases. The modular configuration allows third-party application 428 to use a mix and match approach to building and scaling the underlying third-party application 428 and expanding the capabilities offered by third-party application 428. The third-party application 428 can choose which analytes to monitor and incorporate into a wellness program based on the sensor data.

The sensor control module may further, at step 870, issue an event notification to the third-party application 428 identifying that the sensor data is available. The sensor data can be further transmitted using the sensor control module interface 520.

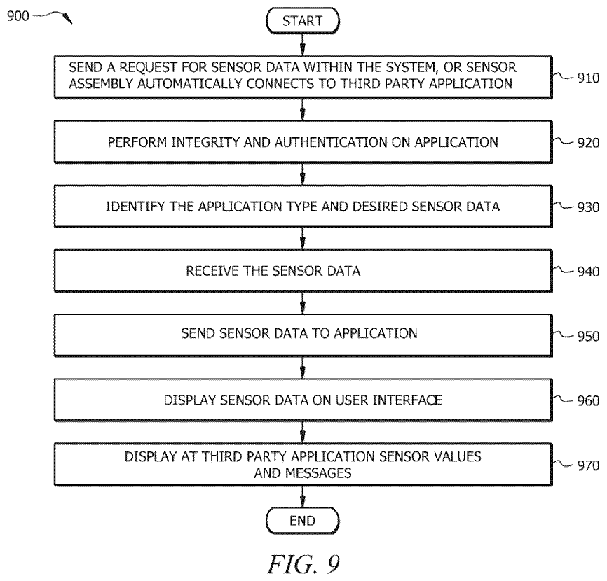

FIG. 9 depicts an example method for communicating sensor data from a sensor to a third-party application 428. As an initial matter, it will be understood by those in the art that any or all of the method steps and/or routines described herein may comprise instructions (e.g., software, firmware, etc.) stored in non-volatile memory of a sensor control device, a remote device (e.g., smartphone, reader), and/or any other computing device that is part of, or in communication with, an analyte monitoring system. Furthermore, the instructions, when executed by the one or more processors of their respective computing device, can cause the one or more processors to perform any one or more of the method steps described herein. Computing device may be the receiving device 200. In addition, although one or more of the method steps and/or routines described herein may comprise software and/or firmware stored on a single computing device, those of skill in the art will recognize that, in certain embodiments, the software and/or firmware may be distributed across multiple similar or disparate computing devices or software modules.

According to one aspect of the embodiments, method 900 can support an application 422, 424, 426 or a third-party application 428 from receiving sensor data for use within the application. At step 910, a third-party application 428 sends a request for sensor data within the system, or the sensor assembly 300 automatically connects to the third-party application 428 using, for example, a BLE connection by issuing a discovery request for BLE capable receiving devices 200 having the third-party application 428. At step 920, the sensor control module 500 verifies the integrity and performs authentication of the third-party application 428. The sensor control module 500 may further communicate with the remote management module 600 to support integrity and obtain content specific information for the third-party application 428. These modules may be available within a software library 400 that a third-party application 428 developer can integrate as a framework within the system of the third-party application 428. At step 930, the sensor control module 500 using logic can identify the third-party application 428 type and desired sensor data to issue requests for the desired data. Optionally, because the session has been authenticated, the sensor assembly 300 may send sensor data through the communication control module 540 and sensor control module interface 520 without a request.

At step 940, the sensor control module 500 receives the sensor data. As stated above, the sensor data can comprise data indicative of an analyte level, such as, for example, a glucose level, a glucose rate-of-change, a glucose trend, or a glucose alarm condition, among others.

At step 950, the sensor data at the sensor control module 500 is sent to the third-party application through the sensor control module interface 520. At step 960, the sensor data is displayed on the user interface 510 of the sensor control module.

At step 970, the third-party application 428 displays any additional messaging related to the sensor assembly 300, including the sensor data relating to analyte levels, notifications, alarms, a message, or other issue regarding the sensors or meal and exercise recommendations based on received sensor data from step 950. Thus, part of the display is via sensor control module 500 regarding analyte levels, whereas another portion of the display on the third-party application 428 is done specifically by the third-party application 428 outside of the control of the sensor control module 500.

The software library 400 and sensor control module 500 as disclosed herein can be used with applications 422, 424, 426. Applications 422, 424, 426 may include various current applications, such as glucose sensor for diabetic monitoring, glucose and ketone sensor for diabetic monitoring, glucose sensor and an insulin delivery device for diabetic monitoring and closed-loop insulin delivery system, and glucose sensor for a wellness application. As disclosed herein, these applications may require various regulated functions and thus need to be submitted in full for regulatory clearance. As disclosed herein, various modifications and functionalities can be added to these applications that do not fall within the core functions for diabetic monitoring and insulin delivery, allowing for unregulated expansion of functions provided by applications based on the sensor data. As further disclosed herein, additional functions can be implemented by applications 422, 424, 426 or third-party applications 428 for wellness, such as glucose sensor for sports or fitness monitoring or for wellness and diet, ketone sensor for wellness or diet plan, such as a keto diet plan, lactate sensor for sports and fitness monitoring, or any number of other applications including alcohol monitoring for treatment and compliance, sST2, Calprotectin, HNL, NT-pro-BNP. Such functionalities can be performed by applications 422, 424, 426 or by third-party applications 428 and reside outside of the core functionality necessary for regulatory review. Thus, enhancements to these functionalities would not need to be submitted for regulatory clearance before introducing the functionalities to the consumer market by use of the modular framework as disclosed herein.

### Alternative Analyte System Configuration

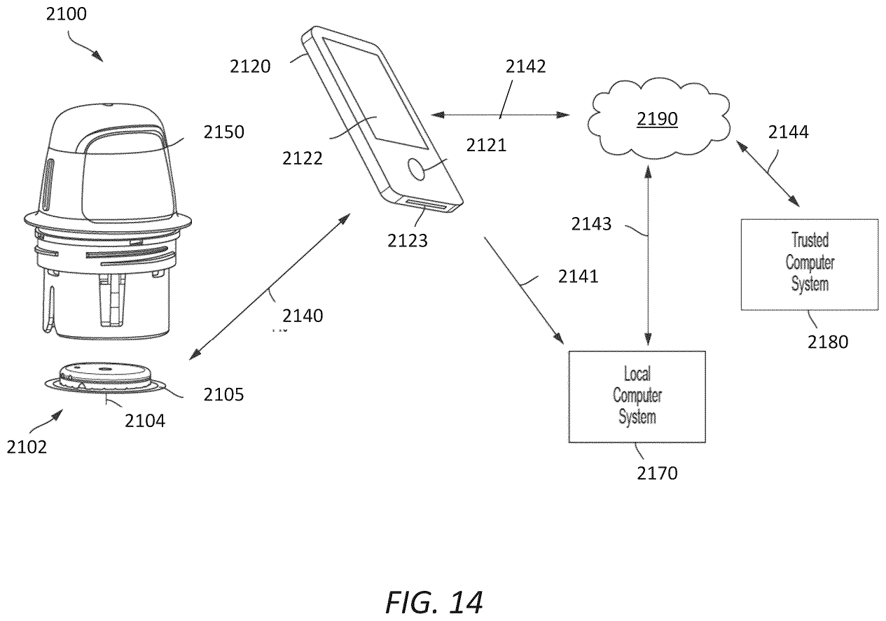

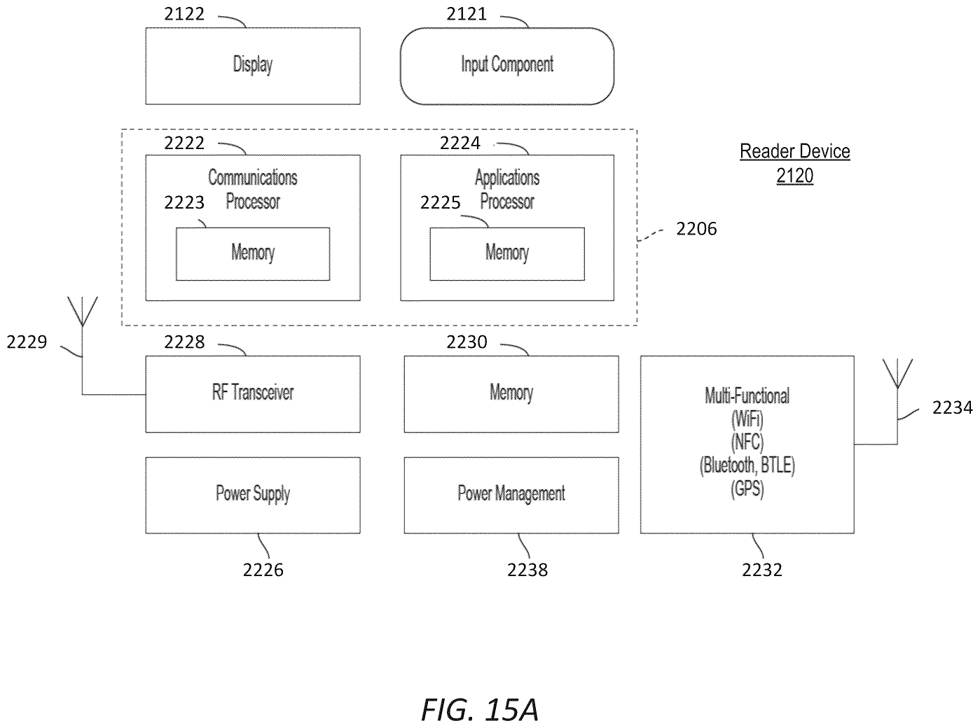

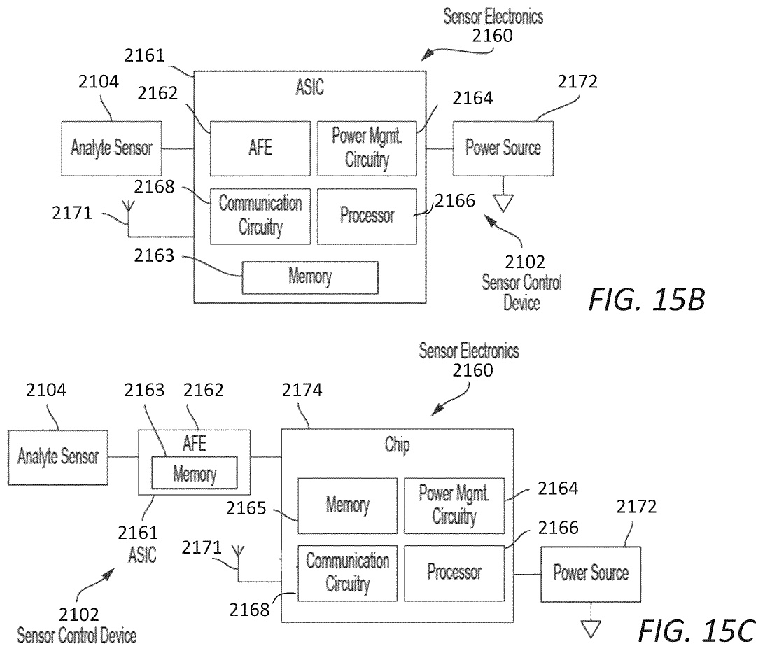

FIG. 14 is a conceptual diagram depicting an example embodiment of an analyte monitoring system 2100 that includes a sensor applicator 2150, a sensor control device 2102, and a reader device 2120. Here, sensor applicator 2150 can be used to deliver sensor control device 2102 to a monitoring location on a user's skin where a sensor 2104 is maintained in position for a period of time by an adhesive patch 2105. Sensor control device 2102, which is further described in FIGS. 15B and 15C, can communicate with reader device 2120 via a communication path 2140 using a wired or wireless technique. Example wireless protocols include Bluetooth, Bluetooth Low Energy (BLE, BTLE, Bluetooth SMART, etc.), Near Field Communication (NFC) and others. Users can view and use applications installed in memory on reader device 2120 using screen 2122 (which, in many embodiments, can comprise a touchscreen), and input 2121. A device battery of reader device 2120 can be recharged using power port 2123. While only one reader device 2120 is shown, sensor control device 2102 can communicate with multiple reader devices 2120. Each of the reader devices 2120 can communicate and share data with one another. More details about reader device 2120 is set forth with respect to FIG. 15A below. Reader device 2120 can communicate with local computer system 2170 via a communication path 2141 using a wired or wireless communication protocol. Local computer system 2170 can include one or more of a laptop, desktop, tablet, phablet, smartphone, set-top box, video game console, or other computing device and wireless communication can include any of a number of applicable wireless networking protocols including Bluetooth, Bluetooth Low Energy (BTLE), Wi-Fi or others. Local computer system 2170 can communicate via communications path 2143 with a network 2190 similar to how reader device 2120 can communicate via a communications path 2142 with network 2190, by a wired or wireless communication protocol as described previously. Network 2190 can be any of a number of networks, such as private networks and public networks, local area or wide area networks, and so forth. A trusted computer system 2180 can include a server and can provide authentication services and secured data storage and can communicate via communications path 2144 with network 2190 by wired or wireless technique.

### Example Embodiment of Reader Device

FIG. 15A is a block diagram depicting an example embodiment of a reader device 2120, which, in some embodiments, can comprise a smartphone. Here, reader device 2120 can include a display 2122, input component 2121, and a processing core 2206 including a communications processor 2222 coupled with memory 2223 and an applications processor 2224 coupled with memory 2225. Also included can be separate memory 2230, RF transceiver 2228 with antenna 2229, and power supply 2226 with power management module 2238. Further, reader device 2120 can also include a multi-functional transceiver 2232, which can communicate over Wi-Fi, NFC, Bluetooth, BTLE, and GPS with an antenna 2234. As understood by one of skill in the art, these components are electrically and communicatively coupled in a manner to make a functional device.

### Example Embodiments of Sensor Control Devices

FIGS. 15B and 15C are block diagrams depicting example embodiments of sensor control devices 102 having an analyte sensor 2104 and sensor electronics 2160 (including analyte monitoring circuitry) that can have the majority of the processing capability for rendering end-result data suitable for display to the user. In FIG. 15B, a single semiconductor chip 2161 is depicted that can be a custom application specific integrated circuit (ASIC). Shown within ASIC 2161 are certain high-level functional units, including an analog front end (AFE) 2162, power management (or control) circuitry 2164, processor 2166, and communication circuitry 2168 (which can be implemented as a transmitter, receiver, transceiver, passive circuit, or otherwise according to the communication protocol). In this embodiment, both AFE 2162 and processor 2166 are used as analyte monitoring circuitry, but in other embodiments either circuit can perform the analyte monitoring function. Processor 2166 can include one or more processors, microprocessors, controllers, and/or microcontrollers, each of which can be a discrete chip or distributed amongst (and a portion of) a number of different chips.

A memory 2163 is also included within ASIC 2161 and can be shared by the various functional units present within ASIC 2161, or can be distributed amongst two or more of them. Memory 2163 can also be a separate chip. Memory 2163 can be volatile and/or non-volatile memory. In this embodiment, ASIC 2161 is coupled with power source 2172, which can be a coin cell battery, or the like. AFE 2162 interfaces with in vivo analyte sensor 2104 and receives measurement data therefrom and outputs the data to processor 2166 in digital form, which in turn processes the data to arrive at the end-result glucose discrete and trend values, etc. This data can then be provided to communication circuitry 2168 for sending, by way of antenna 2171, to reader device 2120 (not shown), for example, where minimal further processing is needed by the resident software application to display the data.

FIG. 15C is similar to FIG. 15B but instead includes two discrete semiconductor chips 2162 and 2174, which can be packaged together or separately. Here, AFE 2162 is resident on ASIC 2161. Processor 2166 is integrated with power management circuitry 2164 and communication circuitry 2168 on chip 2174. AFE 2162 includes memory 2163 and chip 2174 includes memory 2165, which can be isolated or distributed within. In one example embodiment, AFE 2162 is combined with power management circuitry 2164 and processor 2166 on one chip, while communication circuitry 2168 is on a separate chip. In another example embodiment, both AFE 2162 and communication circuitry 2168 are on one chip, and processor 2166 and power management circuitry 2164 are on another chip. It should be noted that other chip combinations are possible, including three or more chips, each bearing responsibility for the separate functions described, or sharing one or more functions for fail-safe redundancy.

### Glucose Wellness Application

In some embodiments, a wellness application that monitors glucose levels in a subject or user may communicate with the sensor control module 500 to obtain glucose data. In other embodiments, the wellness application may communicate with the sensor control device to obtain glucose data. The user may not have diabetes but may want to monitor their glucose levels to improve their health. When a user first opens or logs into the glucose wellness application, a series of GUIs may appear to customize the user's experience and assist the user in making goals for the glucose wellness application to monitor.

### Live Screen

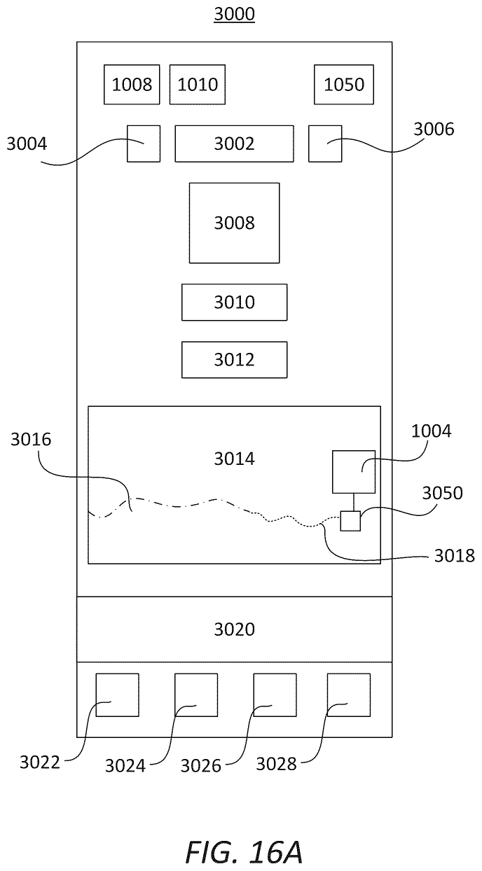

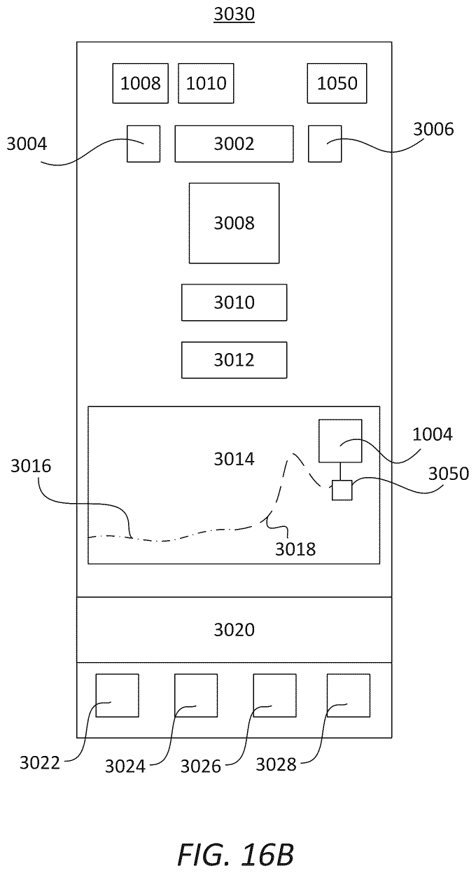

The glucose wellness application may include a live home screen that may include the banner 1002 described elsewhere in this application. As described elsewhere, the banner 1002 may include any of a real time concentration value 1004, a trend arrow 1006, an icon 1008 indicating the status of the biosensor, information 1010 regarding the status of the biosensor, and an additional indication of the biosensor status 1012. In some embodiments, the banner may include the icon 1008 indicating the status of the biosensor, information 1010 regarding the status of the biosensor, and the real time concentration value 1004. As seen in FIGS. 16A-16B, these banner elements may not be located adjacent to each other on the GUI 3000, 3030 for the live glucose wellness screen. For example, the icon 1008 indicating the status of the biosensor and the information 1010 regarding the status of the biosensor may be located at a top of a GUI, while the real time concentration value 1004 may be located in a graph.

As seen in FIGS. 16A-16B, live or home glucose wellness screen GUI 3000, 3030 may include the date 3002 for the current displayed data, along with arrows 3004, 3006 that allow the user to toggle to other days. The glucose wellness live GUI 3000, 3030 may include a graphic 3008 that is color coded with a color that is indicative of a current state of the user's glucose. Alternatively, the glucose wellness live GUI 3000, 3030 may only include a description 3010 of the user's current glucose variability state. The current state of the user's glucose may be based on a user's glucose variability metric. In some embodiments, the graphic 3008 may be a circle. In some embodiments, as described with reference to FIG. 16A, the graphic 3008 may be colored green if the user's current glucose state is steady or in a steady state. In some embodiments, as described with reference to FIG. 16B, the graphic 3008 may be colored orange if the user's current glucose state is unsteady or a spike in glucose levels has been detected.

The glucose wellness live GUI 3000, 3030 (or elsewhere in the application) may include a description 3010 of the user's current glucose variability state, which is based on a metric related to the user's glucose variability. In some embodiments, if the user's current glucose state (variability) is steady or in a steady state, or balanced or in a balanced state, as seen in FIG. 16A, the description may be displayed as “Steady,” “It's Steady,” “Balanced,” or “You're Okay.” In some embodiments, if the user's current glucose variability state (variability) is not steady, not in steady state, unbalanced, not balanced, or a spike is detected, as seen in FIG. 16B, the description may be displayed as “Spike Detected,” “Out of Balance,” “Unbalanced,” “Unsteady,” or “Oh No!”

In some embodiments, the determination of a current glucose variability state, which may be based on a metric related to the user's glucose variability, for a certain window of time may be a binary determination. In some embodiments, the user's glucose variability state may only be one of two options, a positive state with less variability and a negative state with more variability. The states may be determined with reference to a threshold value. In some embodiments, the two options may be steady or unsteady, alternatively steady or spike detected, alternatively steady state or not steady state, alternatively a balanced or an unbalanced state, alternatively “you're okay” vs. “Oh No!” In some embodiments, the user's glucose variability metric for a certain window of time may be a ternary determination. The user's glucose state may only be one of three options.

In some embodiments, a rolling window from the current time or current glucose concentration may be analyzed to determine the user's glucose variability state or glucose variability metric. The rolling window may be computed from a certain timeframe prior to the present timestamp. For example, a rolling window of about 1 hour, alternatively about 1½ hours, alternatively about 2 hours, alternatively about 2½ hours, alternatively about 3 hours, alternatively about 3½ hours, alternatively about 4 hours from a specific time (e.g., the current time or a time point in the past) may be analyzed to determine the user's glucose state. The rolling window may shift periodically. For example, the rolling window may shift every 10 minutes, alternatively every 15 minutes alternatively every 20 minutes, alternatively every 30 minutes, alternatively every 40 minutes, alternatively every 45 minutes alternatively every 50 minutes, alternatively every 60 minutes. The user's glucose state or glucose variability metric may be determined periodically after each shift of the rolling window. For example, a fixed time window (e.g., a 1-hour or 2-hour time window) may shift every 30 minutes and a user's glucose status or glucose variability metric may be determined each 30 minutes. In some embodiments, the rolling window may be constant. In other embodiments, the rolling window may differ or vary throughout a day. In some embodiments, the interval at which the rolling window shifts may be constant. In other embodiments, the interval at which the rolling window shifts may differ or vary throughout a day.

In some embodiments, a user's glucose variability state may be determined to have less variability, i.e., to be steady, in a steady state, or balanced if the user's glucose levels did not vary more than a preset threshold from a running baseline of the rolling window time period. In some embodiments, the running baseline may be an average of the glucose values for the rolling window. In other embodiments, the running baseline may be a median of the glucose values for the rolling window. In some embodiments, the preset threshold may be ±about 20 mg/dl, alternatively ±about 25 mg/dL, alternatively ±about 30 mg/dL, alternatively ±about 35 mg/dL, alternatively ±about 40 mg/dL. In some embodiments, a spike may be detected if glucose levels are determined to be outside of the preset threshold from the baseline. The glucose wellness live GUI 3000, 3030 may also include a link 3012 that the user may tap to see additional details about their current glucose state.

In some embodiments, a user's glucose variability state may be determined to have less variability, i.e., to be steady, in a steady state, or balanced if a difference between a maximum glucose level and a minimum glucose level in a time period was less than a preset threshold. The threshold may be about 20 mg/dL, alternatively about 25 mg/dL, alternatively about 30 mg/dL, alternatively about 35 mg/dL, alternatively about 40 mg/dL.

In other embodiments, a user's current glucose variability state, based on a glucose variability metric, may be determined to be steady, in a steady state, or balanced if the user's glucose levels did not go above a high threshold or below a low threshold, i.e., stayed within a target range, for the rolling window. The user's current glucose variability state may be determined to be balanced or steady if the time in range is above a certain threshold. The threshold when the time in range is being evaluated may be about 100%, alternatively about 95%, alternatively about 90%, alternatively about 85%, alternatively about 80%, alternatively about 75%, alternatively about 70% of the time period. Thus, for a time period of about 2 hours, the variability metric may be balanced (positive) if the user's time in range is above about 84 minutes in range, alternatively above about 90 minutes in range, alternatively above about 96 minutes in range, alternatively above about 102 minutes in range, alternatively above about 108 minutes in range, alternatively above about 114 minutes in range. Alternatively, the user's current glucose variability state, based on a glucose variability metric, may be determined to be balanced or steady if the time out of range is less than a certain threshold. The threshold when the time out of range is being evaluated may be about 5%, alternatively about 10%, alternatively about 15%, alternatively about 20%, alternatively about 25%, alternatively about 30%, alternatively about 35% of the time period. Thus, for a time period of about 2 hours, the variability metric may be balanced (positive) if the user's time out of range is less than about 6 minutes, alternatively less than about 12 minutes, alternatively less than about 18 minutes, alternatively less than about 24 minutes, alternatively less than about 28 minutes, alternatively less than about 32 minutes.

In other embodiments, a user's current glucose variability state, based on a glucose variability metric, may be determined to be steady, in a steady state, or balanced if the user's glucose levels did not go above a high threshold or below a low threshold, i.e., stayed within a target range, for the rolling window, and/or also did not vary more than a preset threshold from a running baseline, as described above. The user may be balanced if the user's glucose levels did not go outside of a target range for more that about 0 minutes, alternatively more than about 5 minutes, alternatively more than about 10 minutes, alternatively more than about 15 minutes, alternatively more than about 20 minutes, alternatively more than about 30 minutes.

The user's glucose variability state or status, based on a glucose variability metric, may be displayed in real time. Each display of the glucose variability state for a rolling window may be replaced by a display of the glucose variability state for the next rolling window evaluated. In some embodiments, a summary or report of the user's glucose state or status may be displayed as a % of a time period, e.g., a % of time of the day, % of time of the week, % of time of a 2-week period, or % of time of the month.

In some embodiments, the user's current glucose state or glucose variability metric may be binary, e.g., may be one of two states. In some embodiments, the binary states are above or below a threshold, within or outside a range, steady and spike detected, alternatively, steady and unsteady, alternatively, balanced and out of balance.

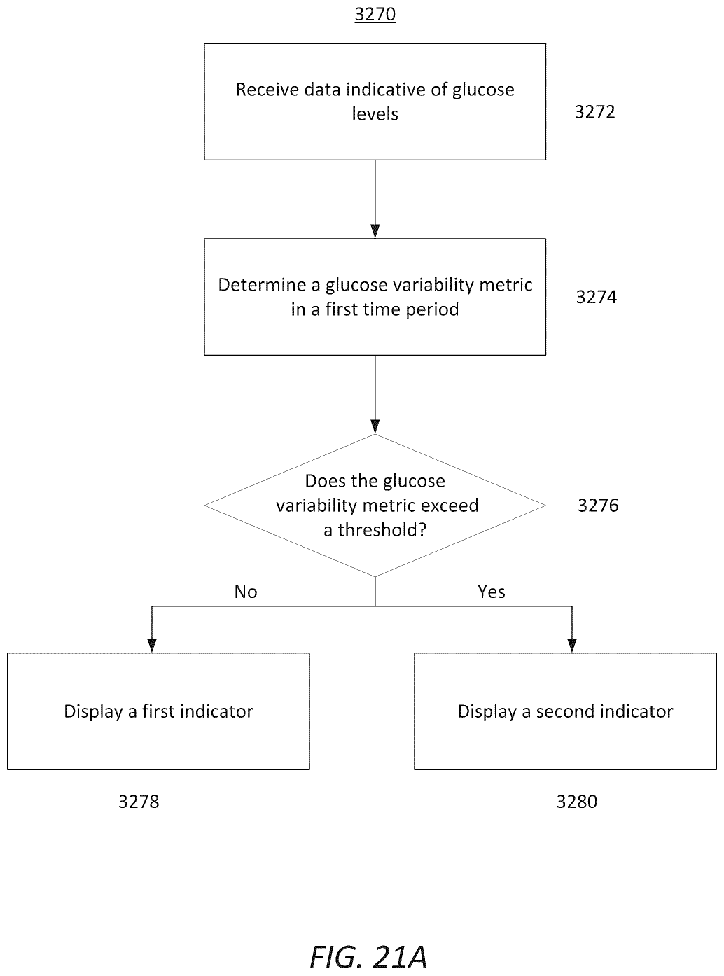

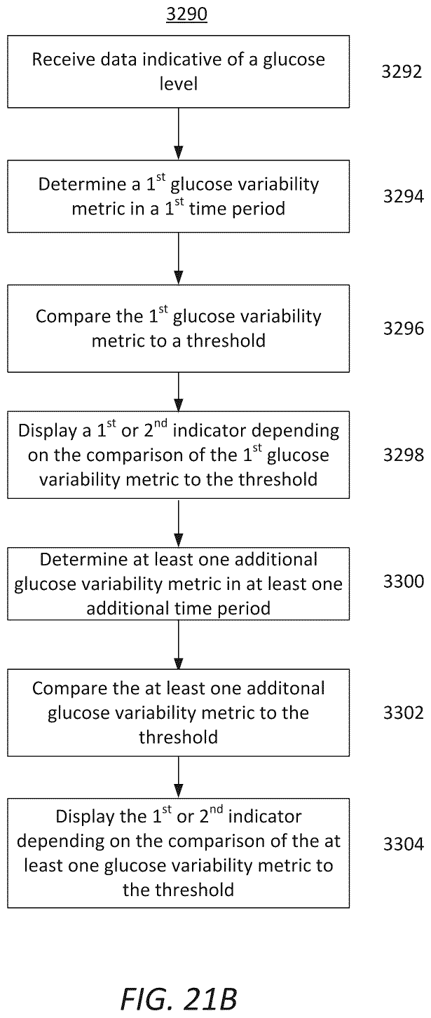

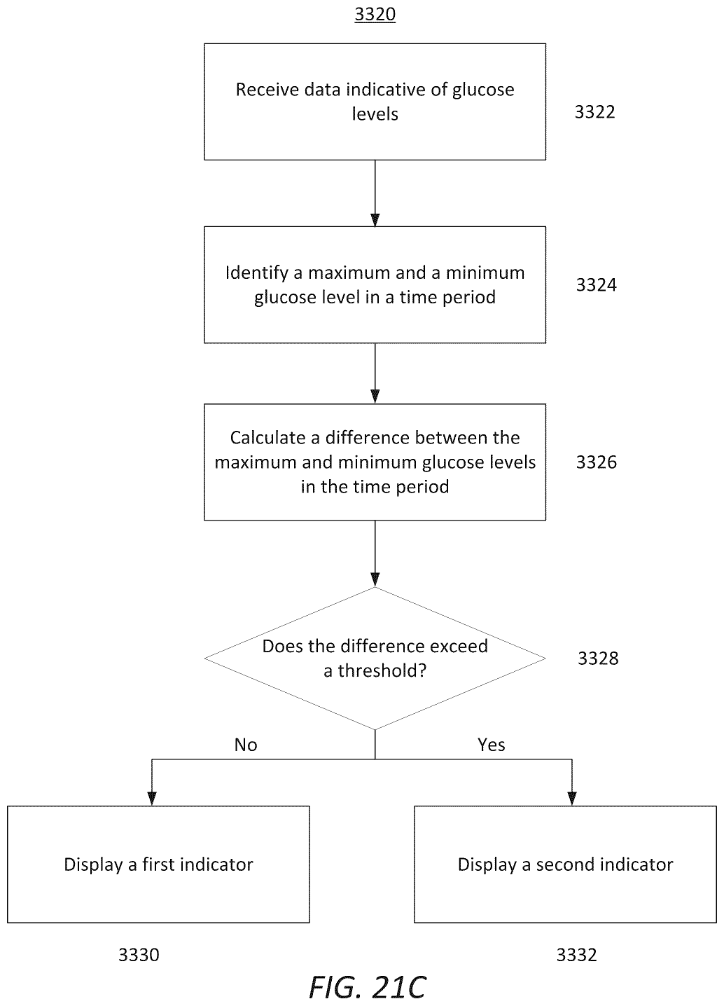

In an exemplary method 3270, as seen in FIG. 21A, a system may receive data indicative of glucose levels in a subject in step 3272. In step 3274, a glucose variability metric may be determined for a first time period from the received glucose data. As described elsewhere, the glucose variability metric may be a measure such as variability with respect to a running baseline, a difference between a maximum and minimum glucose level, time in or out of a target range during the relevant time period, or a combination thereof.

In step 3276, the glucose variability metric may be compared to a threshold value. The threshold may differ depending on the type of glucose variability metric being compared. The threshold when the variability with respect to a running baseline is being evaluated may be ±about 20 mg/dL, alternatively ±about 25 mg/dL, alternatively ±about 30 mg/dL, alternatively ±about 35 mg/dL, alternatively ±about 40 mg/dL. The threshold when the difference between a maximum and a minimum glucose level is being evaluated may be about 20 mg/dL, alternatively about 25 mg/dL, alternatively about 30 mg/dL, alternatively about 35 mg/dL, alternatively about 40 mg/dL. The threshold when the time out of range is being evaluated may be about 5%, alternatively about 10%, alternatively about 15%, alternatively about 20%, alternatively about 25%, alternatively about 30%, alternatively about 35% of the time period. Thus, for a time period of about 2 hours, for a user to be considered in balance, the time out of range threshold may be about 6 minutes, alternatively about 12 minutes, alternatively about 18 minutes, alternatively about 24 minutes, alternatively about 28 minutes, alternatively about 32 minutes.

If the glucose variability metric does not exceed the threshold, then in step 3278, a first indicator may be displayed. The first indicator may be a positive indicator that the subject was balanced, steady, or in steady state for the time period evaluated.

If the glucose variability metric does exceed the threshold, then in step 3280, a second indicator may be displayed. The second indicator may be a negative indicator that the subject was not balanced, unbalanced, not steady, unsteady, or spiky for the time period evaluated.

In another exemplary method 3290, as seen in FIG. 21B, in step 3292, a system may receive data indicative of glucose levels in a subject. In step 3294, a first glucose variability metric may be determined for a first time period from the received glucose data. As described elsewhere, the first glucose variability metric may be a measure such as a difference between a maximum and minimum glucose level, variability with respect to a running baseline, time in or out of a target range during the relevant time period, or combinations thereof.

In step 3296, the first glucose variability metric is compared to a threshold. The threshold may differ depending on the type of glucose variability metric being compared. The threshold when the variability with respect to a running baseline is being evaluated may be ±about 20 mg/dL, alternatively ±about 25 mg/dL, alternatively ±about 30 mg/dL, alternatively ±about 35 mg/dL, alternatively ±about 40 mg/dL. The threshold when the difference between a maximum and a minimum glucose level is being evaluated may be about 20 mg/dL, alternatively about 25 mg/dL, alternatively about 30 mg/dL, alternatively about 35 mg/dL, alternatively about 40 mg/dL. The threshold when the time out of range is being evaluated may be about 5%, alternatively about 10%, alternatively about 15%, alternatively about 20%, alternatively about 25%, alternatively about 30%, alternatively about 35% of the time period. Thus, for a time period of about 2 hours, the time out of range threshold may be about 6 minutes, alternatively about 12 minutes, alternatively about 18 minutes, alternatively about 24 minutes, alternatively about 28 minutes, alternatively about 32 minutes.

In step 3298, a first or a second indicator may be displayed depending on the results of the comparison of the first glucose variability metric to the threshold. For example, if the first glucose variability metric does not exceed the threshold, a first indicator may be displayed. The first indicator may a positive indicator that the subject was balanced, steady, or in steady state for the time period evaluated. If, however, the first glucose variability metric does exceed the threshold, a second indicator may be displayed. The second indicator may be a negative indicator that the subject was not balanced, unbalanced, not steady, unsteady, or spiky for the time period evaluated.

In step 3300, at least one additional glucose variability metric may be determined for at least one additional time period from the received glucose data. As described elsewhere, the at least one additional glucose variability metric may be a measure such as a difference between a maximum and minimum glucose level, variability with respect to a running baseline, time in or out of a target range during the relevant time period, or combinations thereof. In some embodiments, the at least one additional glucose variability metric is the same type as the first glucose variability metric. As described elsewhere, the time period may be a rolling time windows that move in increments of, e.g., 15 minutes, alternatively 30 minutes, alternatively 45 minutes, alternatively 1 hour. The length of the time window may be about 1 hour, alternatively about 2 hours, alternatively about 3 hours, or combinations thereof.

In step 3302, the at least one glucose variability metric is compared to the threshold value. In some embodiments, the threshold is the same threshold compared to the first glucose variability metric. The threshold may differ depending on the type of glucose variability metric being compared. The threshold when the variability with respect to a running baseline is being evaluated may be ±about 20 mg/dL, alternatively ±about 25 mg/dL, alternatively ±about 30 mg/dL, alternatively ±about 35 mg/dL, alternatively ±about 40 mg/dL. The threshold when the difference between a maximum and a minimum glucose level is being evaluated may be about 20 mg/dL, alternatively about 25 mg/dL, alternatively about 30 mg/dL, alternatively about 35 mg/dL, alternatively about 40 mg/dL. The threshold when the time out of range is being evaluated may be about 5%, alternatively about 10%, alternatively about 15%, alternatively about 20%, alternatively about 25%, alternatively about 30%, alternatively about 35% of the time period. Thus, for a time period of about 2 hours, the time out of range threshold may be about 6 minutes out of range, alternatively about 12 minutes out of range, alternatively about 18 minutes out of range, alternatively about 24 minutes out of range, alternatively about 28 minutes out of range, alternatively about 32 minutes out of range.

In step 3304, the first or the second indicator may be displayed depending on the results of the comparison of the at least one additional glucose variability metric. For example, if the at least one additional glucose variability metric does not exceed the threshold, the first indicator may be displayed. The first indicator may a positive indicator that the subject was balanced, steady, or in steady state for the time period evaluated. If, however, the at least one additional glucose variability metric does exceed the threshold, the second indicator may be displayed. The second indicator may be a negative indicator that the subject was not balanced, unbalanced, not steady, unsteady, or spiky for the time period evaluated.

In another exemplary method 3320, as seen in FIG. 21C, a system may receive data indicative of glucose levels in a subject in step 3322. In step 3324, a maximum glucose level and a minimum glucose level may be identified or determined for a time period from the received glucose data. In step 3326, a difference between the maximum and minimum glucose levels may be calculated or determined.

In step 3328, the difference between the maximum and minimum glucose levels may be compared to a threshold value. The threshold may be ±about 20 mg/dL, alternatively ±about 25 mg/dL, alternatively ±about 30 mg/dL, alternatively ±about 35 mg/dL, alternatively ±about 40 mg/dL.

If the calculated difference does not exceed the threshold, then in step 3330, a first indicator may be displayed. The first indicator may a positive indicator that the subject was balanced, steady, or in steady state for the time period evaluated.

If the calculated difference does exceed the threshold, then in step 3332, a second indicator may be displayed. The second indicator may be a negative indicator that the subject was not balanced, unbalanced, not steady, unsteady, or spiky for the time period evaluated.

In some embodiments, in further steps, at least one additional maximum and at least one additional minimum may be identified in at least one additional time period. The difference between the at least one additional maximum and at least one additional minimum may be calculated. The difference may then be compared to a threshold to determine if the difference exceeds or is below the threshold. If the difference between the at least one additional maximum and at least one additional minimum does not exceed the threshold, then the first indicator may be displayed. If the difference between the at least one additional maximum and at least one additional minimum exceeds the threshold, then the second indicator may be displayed.

For all of the methods described, the first or second indicators may be displayed in a graphic, text, or icons, which may optionally be color-coded.

For all of the methods described, in some embodiments, the first or second indicators are displayed in real time after the appropriate indicator is determined for a time period. After the appropriate indicator is determined for the at least one additional time period, the appropriate indicator is displayed, replacing the display of the indicator for the first time period. Each indicator for the next successive rolling time period may then replace the indicator for the previous time period.

The glucose wellness live GUI 3000, 3030 may also include a graph 3014 that includes a glucose curve 3016. The graph 3046 may also highlight the current glucose level with an enlarged marker 3050 on the glucose curve 3016. In some embodiments, the graph 3014 may also include a current glucose level 1004 of the user. The current glucose level may be reported at the end of the glucose curve 3016 over the appropriate time on an x-axis of the graph 3014. The glucose curve 3016 may also include a portion 3018 that has a different color than the rest of the curve. In some embodiments, the portion 3018 with a different color may correspond to the portion of the graph corresponding to the rolling window that is being analyzed to determine the glucose state of the user. In some embodiments, the color of the portion 3018 may be the same color as the graphic 3008 indicating the current glucose state. For example, if the user's current glucose state is “steady,” then the graphic 3008 and the portion 3018 may both be colored green. In other embodiments, if the user's current glucose state is “spike detected” or “not steady state,” then the graphic 3008 and the portion 3018 may both be colored orange.

In some embodiments, the glucose wellness application may have a target concentration range for the user. The target concentration range may be automatically set by the glucose wellness application, or may be manually set by the user or the user's health care provider. In some embodiments, the portion of the glucose curve within the rolling window and within the target range may be colored a first color (e.g., green) and the portion of the glucose curve within the rolling window and outside of the target range may be colored a second color (e.g., orange). The portion of the glucose curve outside of the rolling window time period may be colored a third color (e.g., gray). In some embodiments, the area under the glucose curve may be colored the same color as the glucose curve. In some embodiments, where a portion of the glucose curve is in the rolling window but above the high threshold of the target range, the area under the curve for this portion of the glucose curve above the threshold may be colored two different colors. The portion of the area under the curve between the glucose curve and the high threshold of the target range may be colored the second color (e.g., orange), and the portion of the area under the curve between the high threshold to the low threshold may be colored the first color (e.g., green).

In some embodiments, the glucose wellness live GUI 3000, 3030 may not prominently display the current glucose level in a large font. In some embodiments, the glucose wellness live GUI 3000, 3030 may state the current glucose level in a sentence or phrase, rather than in a large font (e.g., greater than size 16) in a graphic. In some embodiments, the glucose wellness live GUI 3000, 3030 may not include the current glucose level, as too much data may confuse or increase the stress of the user. In some embodiments, the graph 3043 may not include units of concentration for the y-axis so that the user can see the relative glucose levels but not the actual numerical concentrations.

The glucose wellness live GUI 3000, 3030 may also include a card 3020 or a plurality of cards that include links to articles. The articles may contain support information or tips. The article presented in the card 3020 may be chosen to be relevant to the user's current glucose state and/or other information from the user. For example, the articles may be based on goals entered by the user or may be based on past data gathered by the glucose wellness application based on the user's glucose patterns. For example, if the user's current glucose state is “steady,” then the article may provide information about how to stay steady, or how steady glucose helps weight loss. In other embodiments, if the user's current glucose state is “spike detected” or “not steady state,” then the article may provide information about how to avoid spikes.

The glucose wellness live GUI 3000, 3030 may also include a daily tip to assist the user in maintaining steady glucose levels.

The user may also be able to view data from past days in the glucose wellness application. In some embodiments, the user may navigate to a past day by tapping the back arrow 3004. Alternatively, the user may tap on the current date and a calendar may appear in which the user may select the day that they want to view. The calendar may be a drop-down window, a pop-up window, or a separate full screen window. The GUI for a past day may include a graph for the 24-hour period, and may also include statistics for that day. In some embodiments, the statistics may include an amount or percent of time in steady state, e.g., 70% steady, and may also include an amount or percent of time that is the best streak for being steady, e.g., 4.5 hours steady. In some embodiments, the statistics may include a breakdown of the day for the amount or percent of time that the user was steady (“Awesome”), an amount of time that there was a smaller amount of variability (e.g., variability above a first threshold (e.g., ±20 mg/dL), “Nice Effort”), and an amount of time that there was a larger variability (e.g., variability above a second threshold (e.g., ±30 mg/dL) that is larger than the first threshold, needs improvement, “keep it flat!”).

### Insights (See Details) Screen

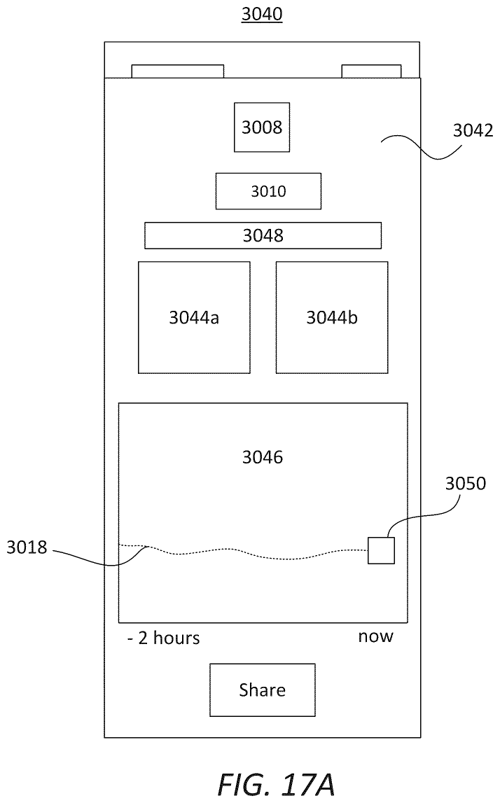

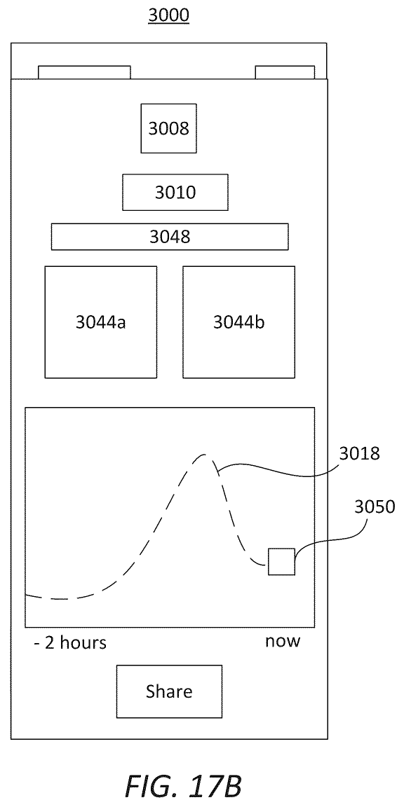

If the user taps or selects the see details link 3012 in FIGS. 16A-16B, a pop-up window GUI 3042, drop-down window GUI, or separate window GUI may appear, as seen in FIGS. 17A-17B. The pop-up window 3042 may include the graphic 3008 and the description 3010 indicating the glucose status of the user, e.g., the user's current glucose status. The pop-up window 3042 may also include further details 3048 regarding the user's glucose status. For example, if the user's current glucose state is “steady” as in FIG. 17A, the further details 3048 may inform the user that they are helping their body be in optimal health. If, however, the user's current glucose state is “spike detected” as in FIG. 17B, the further details 3048 may inform the user that the detected spike may be the cause of hunger, fatigue, and cravings. The further details 3048 may further tell the user not to worry and that their body will bring their glucose level back to normal. The pop-up window 3042 may also include a carousel of cards 3044a-3044b with links to different articles. As explained previously, the articles presented in the carousel 3044a-3044b may be chosen to be relevant to the user's determined or reported glucose state and/or other information from the user.

The pop-up window 3042 may also include a graph 3046 that includes the portion 3018 of the glucose curve that was used to determine the current glucose status. For example, if the rolling window was determined from a 2-hour period, then the graph 3046 may include the last two hours of data. The portion 3018 of the glucose graph may be the same color as the graphic 3008 indicating the current glucose state and as seen in graph 3014. In some embodiments, the graph 3046 may only include a smaller portion of the portion 3018 of the glucose curve that was used to determine the current glucose status. The graph 3046 may also highlight the current glucose level with an enlarged marker 3050 on the glucose curve 3018.

In some embodiments, the glucose wellness application may also display a time that the user spent in a target range, where the target range has a low threshold and a high threshold and the target range is the range between the low and high threshold. The glucose wellness application may report the amount of hours or percentage that the user was in the target range for the day, the week, and/or all time. The glucose wellness application may also report the best streak that the user was in the target range for the day, for the week, and/or for all time.

### Share Screen

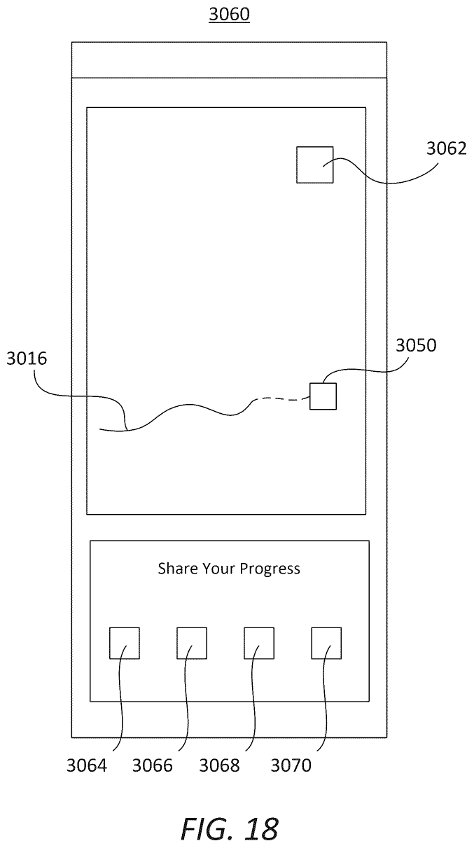

The pop-up window 3042 may also include a share link. If selected, a share GUI 3060 may appear that allows the user to share their progress, as seen in FIG. 18. The share GUI 3060 may include a link 3062 to associate a photo with their progress, either by taking a new photo or associating a photo from a library. The progress to be shared may also include a version of the glucose graph 3016. The user may be able to zoom in and/or crop the graph to only share a portion of the graph. The share GUI 3060 may also include links to share the user's progress through a message 3064 or to third party messaging and social media applications such as Facebook 3066, Instagram 3068, or other options 3070.

Moreover, various GUIs of the glucose wellness application may include a share link 1050 to enable sharing of various data or information.

### Track Screen

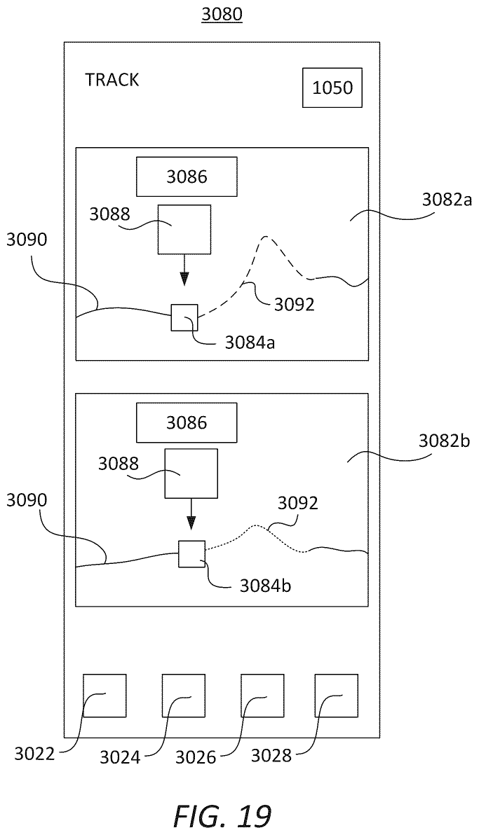

The glucose wellness application may also include a track GUI 3080, as seen in FIG. 19. The track GUI 3808 may include a plurality of cards 3082a-3082b that illustrate the user's glucose response to different conditions. The conditions may include the consumption of different foods and drinks, activity (such as exercise) before and/or after the food/drink consumption, the user's feelings, and other activities. The display of these cards may quickly enable the user to see how their glucose levels were affected by these different activities. In some embodiments, each card 3082a-3082b may include a glucose curve 3090 for a window of time that includes the condition or activity, such as the consumption of food. The window of time may include a portion of time before and after the condition or activity. For example, the window of time may include a 2 hour window, alternatively a 2.5 hour window, alternatively a 3 hour window, alternatively a 3.5 hour window, alternatively a 4 hour window, alternatively a 4.5 hour window, alternatively a 5 hour window. In some embodiments, the window may be chosen such that the condition or activity may have occurred in approximately the first ⅕ of the window, alternatively the first ¼ of the window, alternatively the first ⅓ of the window, alternatively the first ½ of the window.

The glucose curve 3090 may also include a colored portion 3092 may also include a portion 3092 that has a different color than the rest of the curve. In some embodiments, the portion 3092 with a different color may correspond to the portion of the graph corresponding to the user's response to the condition. The colored portion may begin at the time that the condition occurred or was logged and extend for a preset period of time. In some embodiments, the preset period of time may be about 1.5 hours, alternatively about 2 hours, alternatively about 2.5 hours, alternatively about 3 hours, alternatively about 3.5 hours, alternatively about 4 hours. In some embodiments, the color of the portion 3018 may be the same color as the graphic 3008 indicating the glucose state determined for the preset period of time. For example, if the glucose state for the preset period of time is “steady,” then the portion 3092 may be colored green. If, however, a spike was detected in the preset period of time, then the portion 3092 of the graph may be colored orange.

The glucose graph cards 3082a-3082b may each include a glucose curve 3090 and an icon 3084a-3084b along the glucose curve 3090 located at the time at which the condition occurred or was logged, e.g., the time a meal was eaten. The icon may include a graphic related to the condition logged or entered. For example, an icon representing food or drink consumed may be a graphic of a knife and fork, a plate and a drink, a bowl of cereal, etc., and an icon representing exercise may be a running stick figure. The glucose graph cards 3082a-b may also each include a description of the condition 3086, e.g., “fries” if French fries were just eaten, or “Salad+Fries” if a salad was also consumed with the French fries. In some embodiments, the glucose graph cards 3082a-3082b may also each include a picture or pictoral representation 3088 of the type of food consumed or the condition entered. The description 3086 and picture 3088, if present, may be located near the icon 3084a-3084b, e.g., above the icon 3084a-3084b. Each of the glucose graph cards 3082a-3082b may also include a relevant date and time of the condition, e.g., the date and time that the condition or event occurred or was logged.

The plurality of cards 3082a-3082b displayed in the track GUI 3080 may be related. In some embodiments, the plurality of cards 3082a-3082b may all illustrate logged events from the same day or week. In some embodiments, the plurality of cards 3082a-3082b may all illustrate logged events that are related. For example, the logged events may all include the consumption of a certain food, e.g., pizza. Some of the plurality of cards 3082a-3082b may also include, e.g., logged exercise before or after the pizza consumption or a salad consumed with the pizza, such that the user will be able to view how the different circumstances resulted in different glucose responses. Some of the plurality of cards 3082a-3082b may also include a logged feeling or energy level. Thus, the presentation of the plurality of cards 3082a-3082b may illustrate the interplay of compounding events and conditions to the user.

### Learn and Explore

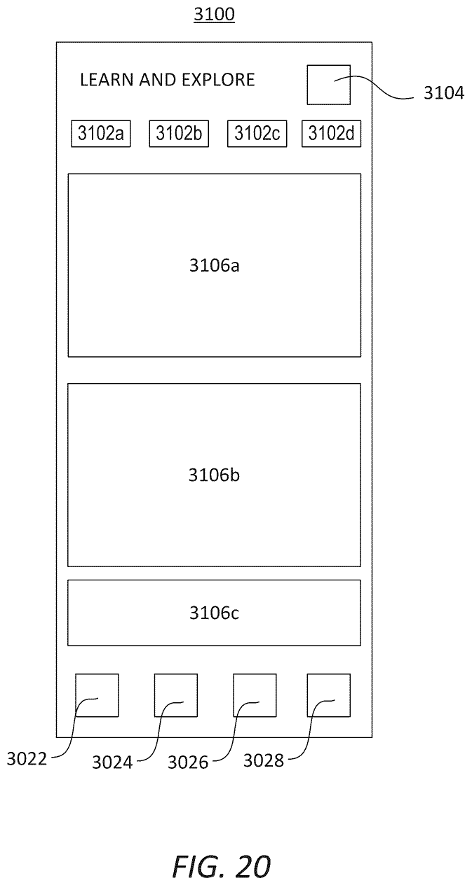

As seen in FIG. 20, the user may tap a link 3026 to view a learn and explore GUI 3100, which provides additional information regarding various relevant topics. Learn and explore GUI 3100 may have a plurality of selectable tabs 3102a-3102d, each tab containing content regarding a different topic of interest. The subject matter of the plurality of selectable tabs 3102a-3102d may include, but are not limited to, content specific to the user (e.g., “for you”), basic content related to glucose wellness (e.g., “basic”), content related to food (e.g., “food”), content related to health (e.g., “health”), content related to lifestyle (e.g., “lifestyle), etc. Learn and explore GUI 3100 may also have a button 3104 that allows the user to bookmark or save the page for easy access at a later time.

Under each selectable tab 3102a-3102d, a plurality of content may be displayed in selectable cards 2022a-2022c, including but not limited to, articles, photographs, text, graphs, tips, and summaries. For example, under the tab specific to the user, the content may include articles explaining how to keep your glucose levels steady, how spikes lead to cravings, and the effects of eating carbs on an empty stomach.

The order of presentation of the content as seen in the displayed selectable cards 3106a-3106c may vary. In some embodiments, the presentation of the order of the content may be randomized in order to maintain a fresh experience for the user. In some embodiments, the presentation of the content may be tailored based on where the user is in their journey. For example, in the beginning of the user experience, content related to getting started, how steady glucose helps weight loss, how spikes lead to cravings, and other basic information may be shown first to the user. As the glucose wellness application gathers more data on the user, e.g., from answers to questions or prompts, from their glucose levels, or from patterns identified in their glucose data, the glucose wellness application may personalize and tailor the content that is presented to the user. The glucose wellness application may also seek input from the user at the end of the article or in another GUI to further personalize their experience.

The glucose wellness application may also include quick links to access different aspects of the application. For instance, at the bottom of each GUI, a link to the live GUI 3022, a link to the track GUI 3024, a link to the Learn GUI 3026, and a link to a settings GUI 3028.

Various aspects of the present subject matter are set forth below, in review of, and/or in supplementation to, the embodiments described thus far, with the emphasis here being on the interrelation and interchangeability of the following embodiments. In other words, an emphasis is on the fact that each feature of the embodiments can be combined with each and every other feature unless explicitly stated otherwise or logically implausible. The embodiments described herein are restated and expanded upon in the following paragraphs without explicit reference to the figures.

In many embodiments, a method for monitoring glucose variability in a subject is described. The method includes the steps of: receiving data indicative of glucose levels of the subject from a sensor control device; determining a first glucose variability metric of the subject in a first time period; comparing the first glucose variability metric to a threshold; and displaying a first indicator if the first glucose variability metric does not exceed the threshold and displaying a second indicator if the first glucose variability metric exceeds the threshold.

In some embodiments, the method further includes the steps of: determining a second glucose variability metric of the subject in a second time period, and wherein the second time period overlaps with the first time period; comparing the second glucose variability metric to the threshold; and displaying the first indicator if the second glucose variability metric does not exceed the threshold and displaying the second indicator if the second glucose variability metric exceeds the threshold. In some embodiments, the second time period starts at a time interval after a start of the first time period. In some embodiments, the time interval is about 30 minutes.

In some embodiments, the first time period and second time period are a same length of total time. In some embodiments, the same length of total time is about 2 hours.

In some embodiments, the first time period and second time period are a different length of total time.

In some embodiments, the first indicator is one of a balanced state or a steady state.

In some embodiments, the second indicator is one of an unbalanced state, an unsteady state, a not steady state, or a spiky state.

In some embodiments, the first glucose variability metric is a binary determination.

In some embodiments, the first glucose variability metric is a difference between a maximum and a minimum glucose level in the first time period.

In some embodiments, the first glucose variability metric is variability with respect to a running baseline of the first time period. In some embodiments, the running baseline is a median of the glucose levels of the first time period. In some embodiments, the running baseline is an average of the glucose levels of the first time period. In some embodiments, the threshold is about ±30 mg/dL.

In some embodiments, the first glucose variability metric is time out of a target glucose range. In some embodiments, the target glucose range is defined by a high glucose threshold and a low glucose threshold. In some embodiments, the threshold is about 20 minutes.

In some embodiments, a display of one of the first or second indicators for the second glucose variability metric replaces a display of the of one of the first or second indicators for the first glucose variability metric.

In some embodiments, the method further includes the steps of: determining a third glucose variability metric of the subject in a third time period, wherein the first, second, and third time periods are the same length of total time, and wherein the third time period overlaps with the second time period; comparing the third glucose variability metric to the threshold; and displaying the first indicator if the third glucose variability metric does not exceed the threshold and displaying the second indicator if the third glucose variability metric exceeds the threshold.

In many embodiments, a system for monitoring glucose variability in a subject includes: an input configured to receive measured glucose data; a display configured to visually present information; and one or more processors coupled with the input, the display, and a memory storing instructions that, when executed by the one or more processors, cause the system to: determine a first glucose variability metric of the subject in a first time period; compare the first glucose variability metric to a threshold; and display a first indicator if the first glucose variability metric does not exceed the threshold and display a second indicator if the first glucose variability metric exceeds the threshold.

In some embodiments, the instructions further cause the one or more processors to: determine a second glucose variability metric of the subject in a second time period, and wherein the second time period overlaps with the first time period; compare the second glucose variability metric to the threshold; and display the first indicator if the second glucose variability metric does not exceed the threshold and displaying the second indicator if the second glucose variability metric exceeds the threshold.

In some embodiments, the first time period and second time period are a same length of total time. In some embodiments, the same length of total time is about 2 hours.

In some embodiments, the first indicator is one of a balanced state or a steady state.

In some embodiments, the second indicator is one of an unbalanced state, an unsteady state, a not steady state, or a spiky state.

In some embodiments, the first glucose variability metric is a binary determination.

In some embodiments, the first glucose variability metric is a difference between a maximum and a minimum glucose level in the first time period.

In some embodiments, the first glucose variability metric is variability with respect to a running baseline of the first time period. In some embodiments, the running baseline is a median of the glucose levels of the first time period. In some embodiments, the running baseline is an average of the glucose levels of the first time period. In some embodiments, the threshold is about ±30 mg/dL.

In some embodiments, the first glucose variability metric is time out of a target glucose range. In some embodiments, the target glucose range is defined by a high glucose threshold and a low glucose threshold. In some embodiments, the threshold is about 20 minutes.

In some embodiments, a display of one of the first or second indicators for the second glucose variability metric replaces a display of the of one of the first or second indicators for the first glucose variability metric.

In some embodiments, the instructions further cause the one or more processors to: determine a third glucose variability metric of the subject in a third time period, wherein the first, second, and third time periods are the same length of total time, and wherein the third time period overlaps with the second time period; compare the third glucose variability metric to the threshold; and display the first indicator if the third glucose variability metric does not exceed the threshold and display the second indicator if the third glucose variability metric exceeds the threshold.

In many embodiments, method for monitoring glucose variability in a subject is described. The method includes the steps of: receiving data indicative of glucose levels of the subject from a sensor control device; identifying a maximum glucose level and a minimum glucose level in a time period; calculating a difference of the maximum glucose level and the minimum glucose level in the time period; comparing the difference to a threshold; and displaying a first indicator if the difference does not exceed the threshold and displaying a second indicator if the difference exceeds the threshold.

In some embodiments, the time period is a first time period and wherein the maximum glucose level and the minimum glucose level are a first maximum glucose level and a second maximum glucose level, respectively, wherein the method further includes the steps of: identifying a second maximum glucose level and a second minimum glucose level in a second time period; calculating a second difference of the second maximum glucose level and the second minimum glucose level in the second time period; comparing the second difference of the second maximum glucose level and the second minimum glucose level in the second time period to the threshold; and displaying a first indicator if the second difference does not exceed the threshold and displaying a second indicator if the second difference exceeds the threshold.

In some embodiments, the time period is about 1 hour.

In some embodiments, the time period is about 2 hours.

In some embodiments, the second time period starts at a time interval after a start of the first time period.

In some embodiments, the time interval is about 30 minutes.

In some embodiments, the second time period overlaps with the first time period.

In some embodiments, the first time period and second time period are a same length of total time.

In some embodiments, the first time period and second time period are a different length of total time.

In some embodiments, the first indicator is one of a balanced state or a steady state.

In some embodiments, the second indicator is one of an unbalanced state, an unsteady state, a not steady state, or a spiky state.

In some embodiments, the threshold is about 30 mg/dL.

In many embodiments, a system for monitoring glucose variability in a subject is described. The system includes: an input configured to receive measured glucose data; a display configured to visually present information; and one or more processors coupled with the input, the display, and a memory storing instructions that, when executed by the one or more processors, cause the system to: identify a maximum glucose level and a minimum glucose level in a time period; calculate a difference of the maximum glucose level and the minimum glucose level in the time period; compare the difference to a threshold; and display a first indicator if the difference does not exceed the threshold and display a second indicator if the difference exceeds the threshold.

In some embodiments, the time period is a first time period and wherein the maximum glucose level and the minimum glucose level are a first maximum glucose level and a second maximum glucose level, respectively, the instructions further cause the one or more processors to: identify a second maximum glucose level and a second minimum glucose level in a second time period; calculate a second difference of the second maximum glucose level and the second minimum glucose level in the second time period; compare the second difference of the second maximum glucose level and the second minimum glucose level in the second time period to the threshold; and display a first indicator if the second difference does not exceed the threshold and displaying a second indicator if the second difference exceeds the threshold.

In some embodiments, the time period is about 1 hour.

In some embodiments, the time period is about 2 hours. In some embodiments, the second time period starts at a time interval after a start of the first time period. In some embodiments, the time interval is about 30 minutes.

In some embodiments, the second time period overlaps with the first time period.

In some embodiments, the first time period and second time period are a same length of total time.

In some embodiments, the first time period and second time period are a different length of total time.

In some embodiments, the first indicator is one of a balanced state or a steady state.

In some embodiments, the second indicator is one of an unbalanced state, an unsteady state, a not steady state, or a spiky state.

In some embodiments, the threshold is about 30 mg/dL.

In many embodiments, a system for displaying metrics relating to a subject is described. The system includes: an input configured to receive measured glucose data; a display configured to visually present information; and one or more processors coupled with the input, the display, and a memory storing instructions that, when executed by the one or more processors, cause the system to: determine a glucose status of the subject based on glucose data received in a rolling window time period; display an indication of the glucose status of the subject in a graphic user interface (GUI), wherein the indication of the glucose status comprises a text description and a graphic having a first color; and display a graph in the GUI, wherein the graph comprises a glucose profile comprising a first portion and a second portion, wherein the first portion and second portion are different colors, and wherein the second portion is the first color.

In some embodiments, the graphic is a circle.

In some embodiments, the glucose status is binary. In some embodiments, the binary glucose status comprises a steady state and an unsteady state. In some embodiments, the binary glucose status comprises a steady state and a state in which a spike is detected.

In some embodiments, the instructions further cause the system to display a current glucose concentration of the subject. In some embodiments, the current glucose concentration of the subject is displayed in the graph.

In some embodiments, the graph further comprises a marker on the glucose profile corresponding to a current glucose level. In some embodiments, the marker is an enlarged circle.

In some embodiments, the GUI does not include a display of a numerical value of a current glucose level of the subject.

In some embodiments, the rolling window time period comprises a preset time interval counting back from a time. In some embodiments, the time is the current time. In some embodiments, the preset time interval is about two hours.

In some embodiments, the glucose status is determined by determining a variability of the glucose data received in the rolling window time period. In some embodiments, the glucose status is determined to be steady if the variability from a running baseline is below a preset threshold. In some embodiments, the preset threshold is ±about 30 mg/dL. In some embodiments, the running baseline is a median glucose concentration of the rolling window time period. In some embodiments, the running baseline is an average glucose concentration of the rolling window time period.

In some embodiments, the glucose status is determined to be unsteady if the variability of the glucose data received in the rolling window time period from a running baseline is above a preset threshold. In some embodiments, the preset threshold is ±about 30 mg/dL. In some embodiments, the running baseline is a median glucose concentration of the rolling window time period. In some embodiments, the running baseline is an average glucose concentration of the rolling window time period.

In some embodiments, the glucose status is determined by analyzing a plurality of glucose concentration levels during the rolling window time period with respect to a target range having a high threshold and a low threshold. In some embodiments, the glucose status is determined to be steady if all of the plurality of glucose concentration levels during the rolling window time period are within the target range. In some embodiments, the glucose status is determined to be unsteady if any of the plurality of glucose concentration levels during the rolling window time period is above the high threshold or below the low threshold.

In some embodiments, the glucose status is determined by (a) analyzing a plurality of glucose concentration levels during the rolling window time period with respect to a target range having a high threshold and a low threshold, and (b) determining a variability of the glucose data received in the rolling window time period with respect to a running baseline.

In some embodiments, the first color is green if the glucose status of the subject is steady.

In some embodiments, the first color is orange if the glucose status of the subject is unsteady.

In some embodiments, the instructions further cause the system to display a plurality of summaries of articles. In some embodiments, the plurality of summaries of articles comprises a plurality of links to the articles. In some embodiments, the plurality of summaries of articles displayed relate to the glucose status of the subject.

In some embodiments, the instructions further cause the system to display a link to a second GUI containing additional details about the glucose status of the subject. In some embodiments, the graph in the GUI is a first graph in a first GUI, and wherein the instructions further cause the system to display the second GUI containing the additional details about the glucose status of the subject in response to a selection of the link.

In some embodiments, the second GUI comprises the indication of the glucose status of the subject and a second graph comprising the second portion of the first graph in the first GUI.

In some embodiments, the second GUI further comprises an additional description related to the glucose status of the subject.

In some embodiments, the second GUI further comprises a plurality of summaries of articles. In some embodiments, the plurality of summaries of articles are displayed in a carousel. In some embodiments, the plurality of summaries of articles displayed relate to the current glucose status of the subject.

In some embodiments, the second GUI further comprises a link to a third GUI to share information related to the glucose status of the subject. In some embodiments, the instructions further cause the system to display the third GUI comprising a graph to be shared and a plurality of links to third party applications. In some embodiments, the graph to be shared is the first graph in the first GUI. In some embodiments, the plurality of links comprises at least one link to a messaging application and at least one link to a social media application. In some embodiments, the third GUI further comprises a link to associate a photograph with the information related to the glucose status of the subject.

In many embodiments, a system for displaying metrics relating to a subject is described. The system includes: an input configured to receive measured glucose data and logged activity information; a display configured to visually present information; and one or more processors coupled with the input, the display, and a memory storing instructions that, when executed by the one or more processors, cause the system to: determine a plurality of glucose statuses of the subject based on glucose data received in a plurality of rolling window time periods, wherein each of the rolling window time periods comprises a logged activity; display a first graph comprising a first glucose profile for a first rolling window time period and a description of a first logged activity, wherein the first glucose profile comprises first, second, and third portions, wherein the first portion and third portions are a first color, and wherein the second portion is a second color, and display a second graph comprising a second glucose profile for a second rolling window time period and a description of a second logged activity, wherein the second glucose profile comprises first, second, and third portions, wherein the first portion and third portions are the first color, and wherein the second portion is a third color.

In some embodiments, the first graph further comprises a first pictoral representation of the first logged activity and the second graph further comprises a second pictoral representation of the second logged activity.

In some embodiments, the second color and the third color are the same color.

In some embodiments, the second color and the third color are different colors.

In some embodiments, the instructions further cause the system to display at least one additional graph comprising at least one additional glucose profile for at least one additional rolling window time period and a description of at least one additional logged activity, wherein the at least one additional glucose profile comprises first, second, and third portions, wherein the first portion and third portions are the first color, and wherein the second portion is the second or third color.

In some embodiments, the first graph further comprises a first icon associated with the first logged activity on the first glucose profile, wherein the first icon is located at the transition from the first portion to the second portion of the first glucose profile.

In some embodiments, the second graph further comprises a second icon associated with the second logged activity on the second glucose profile, wherein the second icon is located at the transition from the first portion to the second portion of the second glucose profile. In some embodiments, the at least one additional graph further comprises at least one additional icon associated with the at least one additional logged activity on the at least one additional glucose profile, wherein the at least one additional icon is located at the transition from the first portion to the second portion of the at least one additional glucose profile.

In some embodiments, the second portion of the first graph is about a 2 hour time period of the first graph.

In some embodiments, the second portion of the second graph is about a 2 hour time period of the second graph. In some embodiments, the second portion of the at least one additional graph is about a 2 hour time period of the at least one additional graph.

In some embodiments, the logged activity information comprises logged food, logged drinks, logged exercise activity, logged feelings, and combinations thereof.

In some embodiments, the first rolling window time period and the second rolling window time period are on the same day.

In some embodiments, the first logged activity and the second logged activity are related.

### CONCLUSION

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. Thus, the foregoing description of specific embodiments of the disclosed subject matter has been presented for purposes of illustration and description. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents. Furthermore, any features, functions, steps, or elements of the embodiments may be recited in or added to the claims, as well as negative limitations that define the inventive scope of the claims by features, functions, steps, or elements that are not within that scope.

### CLAUSES

Exemplary embodiments are set out in the following numbered clauses.

Clause 1. A method for monitoring glucose variability in a subject, comprising:
receiving data indicative of glucose levels of the subject from a sensor control device;
determining a first glucose variability metric of the subject in a first time period;
comparing the first glucose variability metric to a threshold; and
displaying a first indicator if the first glucose variability metric does not exceed the threshold and displaying a second indicator if the first glucose variability metric exceeds the threshold.

Clause 2. The method of clause 1, further comprising the steps of:
determining a second glucose variability metric of the subject in a second time period, and wherein the second time period overlaps with the first time period;
comparing the second glucose variability metric to the threshold; and
displaying the first indicator if the second glucose variability metric does not exceed the threshold and displaying the second indicator if the second glucose variability metric exceeds the threshold.

Clause 3. The method of clause 2, wherein the second time period starts at a time interval after a start of the first time period.

Clause 4. The method of clause 3, wherein the time interval is about 30 minutes.

Clause 5. The method of clause 2, wherein the first time period and second time period are a same length of total time.

Clause 6. The method of clause 5, wherein the same length of total time is about 2 hours.

Clause 7. The method of clause 2, wherein the first time period and second time period are a different length of total time.

Clause 8. The method of clause 1, wherein the first indicator is one of a balanced state or a steady state.

Clause 9. The method of clause 1, wherein the second indicator is one of an unbalanced state, an unsteady state, a not steady state, or a spiky state.

Clause 10. The method of clause 1, wherein the first glucose variability metric is a binary determination.

Clause 11. The method of clause 1, wherein the first glucose variability metric is a difference between a maximum and a minimum glucose level in the first time period.

Clause 12. The method of clause 1, wherein the first glucose variability metric is variability with respect to a running baseline of the first time period.

Clause 13. The method of clause 12, wherein the running baseline is a median of the glucose levels of the first time period.

Clause 14. The method of clause 12, wherein the running baseline is an average of the glucose levels of the first time period.

Clause 15. The method of clause 12, wherein the threshold is about ±30 mg/dL.

Clause 16. The method of clause 1, wherein the first glucose variability metric is time out of a target glucose range.

Clause 17. The method of clause 16, wherein the target glucose range is defined by a high glucose threshold and a low glucose threshold.

Clause 18. The method of clause 16, wherein the threshold is about 20 minutes.

Clause 19. The method of clause 2, wherein a display of one of the first or second indicators for the second glucose variability metric replaces a display of the of one of the first or second indicators for the first glucose variability metric.

Clause 20. The method of clause 1, further comprising the steps of:
determining a third glucose variability metric of the subject in a third time period, wherein the first, second, and third time periods are the same length of total time, and wherein the third time period overlaps with the second time period;
comparing the third glucose variability metric to the threshold; and
displaying the first indicator if the third glucose variability metric does not exceed the threshold and displaying the second indicator if the third glucose variability metric exceeds the threshold.

Clause 21. A system for monitoring glucose variability in a subject, the system comprising:
an input configured to receive measured glucose data;
a display configured to visually present information; and
one or more processors coupled with the input, the display, and a memory storing instructions that, when executed by the one or more processors, cause the system to:
determine a first glucose variability metric of the subject in a first time period;
compare the first glucose variability metric to a threshold; and
display a first indicator if the first glucose variability metric does not exceed the threshold and display a second indicator if the first glucose variability metric exceeds the threshold.

Clause 22. The system of clause 21, wherein the instructions further cause the one or more processors to:
determine a second glucose variability metric of the subject in a second time period, and wherein the second time period overlaps with the first time period;
compare the second glucose variability metric to the threshold; and
display the first indicator if the second glucose variability metric does not exceed the threshold and displaying the second indicator if the second glucose variability metric exceeds the threshold.

Clause 23. The system of clause 22, wherein the same length of total time is about 2 hours.

Clause 24. The system of clause 22, wherein the first time period and second time period are a same length of total time.

Clause 25. The system of clause 21, wherein the first indicator is one of a balanced state or a steady state.

Clause 26. The system of clause 21, wherein the second indicator is one of an unbalanced state, an unsteady state, a not steady state, or a spiky state.

Clause 27. The system of clause 21, wherein the first glucose variability metric is a binary determination.

Clause 28. The system of clause 21, wherein the first glucose variability metric is a difference between a maximum and a minimum glucose level in the first time period.

Clause 29. The system of clause 21, wherein the first glucose variability metric is variability with respect to a running baseline of the first time period.

Clause 30. The system of clause 28, wherein the running baseline is a median of the glucose levels of the first time period.

Clause 31. The system of clause 28, wherein the running baseline is an average of the glucose levels of the first time period.

Clause 32. The system of clause 28, wherein the threshold is about ±30 mg/dL.

Clause 33. The system of clause 21, wherein the first glucose variability metric is time out of a target glucose range.

Clause 34. The system of clause 32, wherein the target glucose range is defined by a high glucose threshold and a low glucose threshold.

Clause 35. The system of clause 32, wherein the threshold is about 20 minutes.

Clause 36. The system of clause 22, wherein a display of one of the first or second indicators for the second glucose variability metric replaces a display of the of one of the first or second indicators for the first glucose variability metric.

Clause 37. The system of clause 22, wherein the instructions further cause the one or more processors to:
determine a third glucose variability metric of the subject in a third time period, wherein the first, second, and third time periods are the same length of total time, and wherein the third time period overlaps with the second time period;
compare the third glucose variability metric to the threshold; and
display the first indicator if the third glucose variability metric does not exceed the threshold and display the second indicator if the third glucose variability metric exceeds the threshold.

Clause 38. A method for monitoring glucose variability in a subject, comprising: receiving data indicative of glucose levels of the subject from a sensor control device;
identifying a maximum glucose level and a minimum glucose level in a time period;
calculating a difference of the maximum glucose level and the minimum glucose level in the time period;
comparing the difference to a threshold; and
displaying a first indicator if the difference does not exceed the threshold and displaying a second indicator if the difference exceeds the threshold.

Clause 39. The method of clause 37, wherein the time period is a first time period and wherein the maximum glucose level and the minimum glucose level are a first maximum glucose level and a second maximum glucose level, respectively, wherein the method further comprises the steps of:
identifying a second maximum glucose level and a second minimum glucose level in a second time period;
calculating a second difference of the second maximum glucose level and the second minimum glucose level in the second time period;
comparing the second difference of the second maximum glucose level and the second minimum glucose level in the second time period to the threshold; and
displaying a first indicator if the second difference does not exceed the threshold and displaying a second indicator if the second difference exceeds the threshold.

Clause 40. The method of clause 37, wherein the time period is about 1 hour.

Clause 41. The method of clause 37, wherein the time period is about 2 hours.

Clause 42. The method of clause 38, wherein the second time period starts at a time interval after a start of the first time period.

Clause 43. The method of clause 42, wherein the time interval is about 30 minutes.

Clause 44. The method of clause 38, wherein the second time period overlaps with the first time period.

Clause 45. The method of clause 38, wherein the first time period and second time period are a same length of total time.

Clause 46. The method of clause 38, wherein the first time period and second time period are a different length of total time.

Clause 47. The method of clause 37, wherein the first indicator is one of a balanced state or a steady state.

Clause 48. The method of clause 37, wherein the second indicator is one of an unbalanced state, an unsteady state, a not steady state, or a spiky state.

Clause 49. The method of clause 37, wherein the threshold is about 30 mg/dL.

Clause 50. A system for monitoring glucose variability in a subject, the system comprising:
an input configured to receive measured glucose data;
a display configured to visually present information; and
one or more processors coupled with the input, the display, and a memory storing instructions that, when executed by the one or more processors, cause the system to:
identify a maximum glucose level and a minimum glucose level in a time period;
calculate a difference of the maximum glucose level and the minimum glucose level in the time period;
compare the difference to a threshold; and
display a first indicator if the difference does not exceed the threshold and display a second indicator if the difference exceeds the threshold.

Clause 51. The system of clause 49, wherein the time period is a first time period and wherein the maximum glucose level and the minimum glucose level are a first maximum glucose level and a second maximum glucose level, respectively, the instructions further cause the one or more processors to:
identify a second maximum glucose level and a second minimum glucose level in a second time period;
calculate a second difference of the second maximum glucose level and the second minimum glucose level in the second time period;
compare the second difference of the second maximum glucose level and the second minimum glucose level in the second time period to the threshold; and
display a first indicator if the second difference does not exceed the threshold and displaying a second indicator if the second difference exceeds the threshold.

Clause 52. The system of clause 49, wherein the time period is about 1 hour.

Clause 53. The system of clause 49, wherein the time period is about 2 hours.

Clause 54. The system of clause 50, wherein the second time period starts at a time interval after a start of the first time period.

Clause 55. The system of clause 53, wherein the time interval is about 30 minutes.

Clause 56. The system of clause 50, wherein the second time period overlaps with the first time period.

Clause 57. The system of clause 50, wherein the first time period and second time period are a same length of total time.

Clause 58. The system of clause 50, wherein the first time period and second time period are a different length of total time.

Clause 59. The system of clause 49, wherein the first indicator is one of a balanced state or a steady state.

Clause 60. The system of clause 49, wherein the second indicator is one of an unbalanced state, an unsteady state, a not steady state, or a spiky state.

Clause 61. The system of clause 49, wherein the threshold is about 30 mg/dL.

Clause 62. A system for displaying metrics relating to a subject, the system comprising:
an input configured to receive measured glucose data;
a display configured to visually present information; and
one or more processors coupled with the input, the display, and a memory storing instructions that, when executed by the one or more processors, cause the system to:
determine a glucose status of the subject based on glucose data received in a rolling window time period;
display an indication of the glucose status of the subject in a graphic user interface (GUI), wherein the indication of the glucose status comprises a text description and a graphic having a first color; and
display a graph in the GUI, wherein the graph comprises a glucose profile comprising a first portion and a second portion, wherein the first portion and second portion are different colors, and wherein the second portion is the first color.

Clause 63. The system of clause 61, wherein the graphic is a circle.

Clause 64. The system of clause 61, wherein the glucose status is binary.

Clause 65. The system of clause 63, wherein the binary glucose status comprises a steady state and an unsteady state.

Clause 66. The system of clause 63, wherein the binary glucose status comprises a steady state and a state in which a spike is detected.

Clause 67. The system of clause 61, wherein the instructions further cause the system to display a current glucose concentration of the subject.

Clause 68. The system of clause 66, wherein the current glucose concentration of the subject is displayed in the graph.

Clause 69. The system of clause 61, wherein the graph further comprises a marker on the glucose profile corresponding to a current glucose level.

Clause 70. The system of clause 68, wherein the marker is an enlarged circle.

Clause 71. The system of clause 61, wherein the GUI does not include a display of a numerical value of a current glucose level of the subject.

Clause 72. The system of clause 61, wherein the rolling window time period comprises a preset time interval counting back from a time.

Clause 73. The system of clause 71, wherein the time is the current time.

Clause 74. The system of clause 71, wherein the preset time interval is about two hours.

Clause 75. The system of clause 61, wherein the glucose status is determined by determining a variability of the glucose data received in the rolling window time period.

Clause 76. The system of clause 74, wherein the glucose status is determined to be steady if the variability from a running baseline is below a preset threshold.

Clause 77. The system of clause 75, wherein the preset threshold is ±about 30 mg/dL.

Clause 78. The system of clause 75, wherein the running baseline is a median glucose concentration of the rolling window time period.

Clause 79. The system of clause 75, wherein the running baseline is an average glucose concentration of the rolling window time period.

Clause 80. The system of clause 74, wherein the glucose status is determined to be unsteady if the variability of the glucose data received in the rolling window time period from a running baseline is above a preset threshold.

Clause 81. The system of clause 79, wherein the preset threshold is ±about 30 mg/dL.

Clause 82. The system of clause 79, wherein the running baseline is a median glucose concentration of the rolling window time period.

Clause 83. The system of clause 79, wherein the running baseline is an average glucose concentration of the rolling window time period.

Clause 84. The system of clause 61, wherein the glucose status is determined by analyzing a plurality of glucose concentration levels during the rolling window time period with respect to a target range having a high threshold and a low threshold.

Clause 85. The system of clause 83, wherein the glucose status is determined to be steady if all of the plurality of glucose concentration levels during the rolling window time period are within the target range.

Clause 86. The system of clause 83, wherein the glucose status is determined to be unsteady if any of the plurality of glucose concentration levels during the rolling window time period is above the high threshold or below the low threshold.

Clause 87. The system of clause 61, wherein the glucose status is determined by (a) analyzing a plurality of glucose concentration levels during the rolling window time period with respect to a target range having a high threshold and a low threshold, and (b) determining a variability of the glucose data received in the rolling window time period with respect to a running baseline.

Clause 88. The system of clause 61, wherein the first color is green if the glucose status of the subject is steady.

Clause 89. The system of clause 61, wherein the first color is orange if the glucose status of the subject is unsteady.

Clause 90. The system of clause 61, wherein the instructions further cause the system to display a plurality of summaries of articles.

Clause 91. The system of clause 89, wherein the plurality of summaries of articles comprises a plurality of links to the articles.

Clause 92. The system of clause 89, wherein the plurality of summaries of articles displayed relate to the glucose status of the subject.

Clause 93. The system of clause 61, wherein the instructions further cause the system to display a link to a second GUI containing additional details about the glucose status of the subject.

Clause 94. The system of clause 92, wherein the graph in the GUI is a first graph in a first GUI, and wherein the instructions further cause the system to display the second GUI containing the additional details about the glucose status of the subject in response to a selection of the link.

Clause 95. The system of clause 93, wherein the second GUI comprises the indication of the glucose status of the subject and a second graph comprising the second portion of the first graph in the first GUI.

Clause 96. The system of clause 94, wherein the second GUI further comprises an additional description related to the glucose status of the subject.

Clause 97. The system of clause 94, wherein the second GUI further comprises a plurality of summaries of articles.

Clause 98. The system of clause 96, wherein the plurality of summaries of articles are displayed in a carousel.

Clause 99. The system of clause 96, wherein the plurality of summaries of articles displayed relate to the current glucose status of the subject.

Clause 100. The system of clause 93, wherein the second GUI further comprises a link to a third GUI to share information related to the glucose status of the subject.

Clause 101. The system of clause 99, wherein the instructions further cause the system to display the third GUI comprising a graph to be shared and a plurality of links to third party applications.

Clause 102. The system of clause 100, wherein the graph to be shared is the first graph in the first GUI.

Clause 103. The system of clause 100, wherein the plurality of links comprises at least one link to a messaging application and at least one link to a social media application.

Clause 104. The system of clause 100, wherein the third GUI further comprises a link to associate a photograph with the information related to the glucose status of the subject.

Clause 105. A system for displaying metrics relating to a subject, the system comprising:
an input configured to receive measured glucose data and logged activity information;
a display configured to visually present information; and
one or more processors coupled with the input, the display, and a memory storing instructions that, when executed by the one or more processors, cause the system to:
   determine a plurality of glucose statuses of the subject based on glucose data received in a plurality of rolling window time periods, wherein each of the rolling window time periods comprises a logged activity;
   display a first graph comprising a first glucose profile for a first rolling window time period and a description of a first logged activity, wherein the first glucose profile comprises first, second, and third portions, wherein the first portion and third portions are a first color, and wherein the second portion is a second color, and
   display a second graph comprising a second glucose profile for a second rolling window time period and a description of a second logged activity, wherein the second glucose profile comprises first, second, and third portions, wherein the first portion and third portions are the first color, and wherein the second portion is a third color.

Clause 106. The system of clause 104, wherein the first graph further comprises a first pictoral representation of the first logged activity and the second graph further comprises a second pictoral representation of the second logged activity.

Clause 107. The system of clause 104, wherein the second color and the third color are the same color.

Clause 108. The system of clause 104, wherein the second color and the third color are different colors.

Clause 109. The system of clause 104, wherein the instructions further cause the system to display at least one additional graph comprising at least one additional glucose profile for at least one additional rolling window time period and a description of at least one additional logged activity, wherein the at least one additional glucose profile comprises first, second, and third portions, wherein the first portion and third portions are the first color, and wherein the second portion is the second or third color.

Clause 110. The system of clause 104, wherein the first graph further comprises a first icon associated with the first logged activity on the first glucose profile, wherein the first icon is located at the transition from the first portion to the second portion of the first glucose profile.

Clause 111. The system of clause 104, wherein the second graph further comprises a second icon associated with the second logged activity on the second glucose profile, wherein the second icon is located at the transition from the first portion to the second portion of the second glucose profile.

Clause 112. The system of clause 108, wherein the at least one additional graph further comprises at least one additional icon associated with the at least one additional logged activity on the at least one additional glucose profile, wherein the at least one additional icon is located at the transition from the first portion to the second portion of the at least one additional glucose profile.

Clause 113. The system of clause 104, wherein the second portion of the first graph is about a 2 hour time period of the first graph.

Clause 114. The system of clause 104, wherein the second portion of the second graph is about a 2 hour time period of the second graph.

Clause 115. The system of clause 111, wherein the second portion of the at least one additional graph is about a 2 hour time period of the at least one additional graph.

Clause 116. The system of clause 104, wherein the logged activity information comprises logged food, logged drinks, logged exercise activity, logged feelings, and combinations thereof.

Clause 117. The system of clause 104, wherein the first rolling window time period and the second rolling window time period are on the same day.

Clause 118. The system of clause 104, wherein the first logged activity and the second logged activity are related.

1-104. (canceled)

105. A system for displaying metrics relating to a subject, the system comprising:
wireless communication circuitry configured to receive measured glucose data and logged activity information;
a display configured to visually present information; and
one or more processors coupled with the wireless communication circuitry, the display, and a memory storing instructions that, when executed by the one or more processors, cause the one or more processors to:
determine a plurality of glucose statuses of the subject based on glucose data received in a plurality of rolling window time periods, wherein each of the rolling window time periods comprises a logged activity;
display a first graph comprising a first glucose profile for a first rolling window time period and a description of a first logged activity, wherein the first glucose profile comprises first, second, and third portions, wherein the first portion and third portions are a first color, and wherein the second portion is a second color, and
display a second graph comprising a second glucose profile for a second rolling window time period and a description of a second logged activity, wherein the second glucose profile comprises first, second, and third portions, wherein the first portion and third portions are the first color, and wherein the second portion is a third color.

106. The system of claim 105, wherein the first graph further comprises a first pictoral representation of the first logged activity and the second graph further comprises a second pictoral representation of the second logged activity.

107. The system of claim 105, wherein the second color and the third color are the same color.

108. The system of claim 105, wherein the second color and the third color are different colors.

109. The system of claim 105, wherein the instructions further cause the one or more processors to display at least one additional graph comprising at least one additional glucose profile for at least one additional rolling window time period and a description of at least one additional logged activity, wherein the at least one additional glucose profile comprises first, second, and third portions, wherein the first portion and third portions are the first color, and wherein the second portion is the second or third color.

110. The system of claim 105, wherein the first graph further comprises a first icon associated with the first logged activity on the first glucose profile, wherein the first icon is located at the transition from the first portion to the second portion of the first glucose profile.

111. The system of claim 105, wherein the second graph further comprises a second icon associated with the second logged activity on the second glucose profile, wherein the second icon is located at the transition from the first portion to the second portion of the second glucose profile.

112. The system of claim 109, wherein the at least one additional graph further comprises at least one additional icon associated with the at least one additional logged activity on the at least one additional glucose profile, wherein the at least one additional icon is located at the transition from the first portion to the second portion of the at least one additional glucose profile.

113. The system of claim 105, wherein the second portion of the first graph is about a 2 hour time period of the first graph.

114. The system of claim 105, wherein the second portion of the second graph is about a 2 hour time period of the second graph.

115. The system of claim 112, wherein the second portion of the at least one additional graph is about a 2 hour time period of the at least one additional graph.

116. The system of claim 105, wherein the logged activity information comprises logged food, logged drinks, logged exercise activity, logged feelings, and combinations thereof.

117. The system of claim 105, wherein the first rolling window time period and the second rolling window time period are on the same day.

118. The system of claim 105, wherein the first logged activity and the second logged activity are related.
